(19) United States
(12) Patent Application Publication
(10) Pub. No.: US 2025/0198980 A1
(43) Pub. Date: Jun. 19, 2025

(54) COMBINATORIAL HIGH-THROUGHPUT SCREENING OF COMPLEX POLYMERIC ENZYME IMMOBILIZATION SUPPORTS

(71) Applicant: The Regents of the University of Colorado, a body corporate, Denver, CO (US)

(72) Inventors: Daniel K. Schwartz, Boulder, CO (US); Joel L. Kaar, Lafayette, CO (US); Héctor Sánchez-Morán, Boulder, CO (US)

(73) Assignee: The Regents of the University of Colorado, a body corporate, Denver, CO (US)

(21) Appl. No.: 18/981,195

(22) Filed: Dec. 13, 2024

Related U.S. Application Data
(60) Provisional application No. 63/609,705, filed on Dec. 13, 2023.

(51) Int. Cl. G01N 33/44; C12N 11/098; C12Q 1/25; C12Q 1/28; C12Q 1/32; C12Q 1/37; C12Q 1/42; C12Q 1/46; C12Q 1/48; C12Q 1/527; G01N 21/64; G01N 21/78

(52) U.S. Cl. CPC G01N 33/442; C12N 11/098; C12Q 1/25; C12Q 1/28; C12Q 1/32; C12Q 1/37; C12Q 1/42; C12Q 1/46; C12Q 1/48; C12Q 1/527; G01N 21/6428; G01N 21/78; G01N 2333/90245; G01N 2333/90661; G01N 2333/90688; G01N 2333/908; G01N 2333/91188; G01N 2333/916; G01N 2333/918; G01N 2333/96433; G01N 2333/974; G01N 2333/976; G01N 2333/984; G01N 2333/988; G01N 2333/992

(57) ABSTRACT

A novel combinatorial and high-throughput platform that enables rapid screening of complex and heterogeneous copolymer brushes as enzyme immobilization supports named Combinatorial High-throughput Enzyme Support Screening (CHESS). Using a 384 well-plate format, we synthesized arrays of three-component polymer brushes in the microwells using photo-activated surface-initiated polymerization, and immobilized enzymes in situ. The utility of CHESS to identify optimal immobilization supports under thermally and chemically denaturing conditions was demonstrated using Bacillus subtilis Lipase A (LipA). The identification of supports with optimal compositions was validated by immobilizing LipA on polymer-brush modified biocatalyst particles. We further demonstrated that CHESS could be used to predict the optimal composition of polymer brushes a priori for the previously unexplored enzyme, alkaline phosphatase (AlkP). Our findings demonstrate that CHESS represents a predictable and reliable platform for dramatically accelerating the search of chemical compositions for immobilization supports and further facilitate the discovery of biocompatible and stabilizing materials.

### CROSS-REFERENCE TO RELATED APPLICATIONS

This application claims the benefit of U.S. Provisional Application No. 63/609,705, filed Dec. 13, 2024.

### STATEMENT OF GOVERNMENT INTEREST

This invention was made with government support under grant number 2103647 awarded by the National Science Foundation. The government has certain rights in the invention.

### FIELD OF INVENTION

This invention relates to methods and platforms to generate large numbers of copolymer brush variants of different compositions to be used as immobilization supports for enzymes in a well-plate format.

### BACKGROUND OF THE INVENTION

Immobilization on optimally designed synthetic supports can provide enzymes with remarkable properties, including dramatically enhanced stability against thermal and chemical deactivation, while also increasing enzyme activity and the improving the ease of recycling. Owing to these advantages, enzyme immobilization has gained widespread interest in many fields, including chemical and pharmaceutical production, biosensing, and bioremediation. Additionally, a growing emphasis on sustainability is driving a shift towards enzyme-based industrial bioprocesses to reduce the use of supported metals and organocatalysts. Recent breakthroughs have underscored the remarkable potential of complex heterogeneous and dynamic materials, including lipid bilayers and copolymer brushes as robust supports for enzyme immobilization. These materials can confer exceptional characteristics to enzymes in a tailor-made manner, via their chemical and physical tunability. This tunability allows for the introduction of diverse chemical moieties that can interact favorably with the surface of the enzyme. For instance, dynamic polymer brushes are thought to preferentially self-assemble around regions on proteins of similar chemical properties. These transient enzyme-polymer interactions can facilitate refolding—akin to a synthetic molecular chaperone—as well as inhibit unfolding. The introduction of new types of non-covalent interactions between enzymes and such materials opens unexplored opportunities for the discovery of novel immobilization supports, which can enable the supra-biological performance of enzymes under industrially relevant process conditions.

Because the composition of an optimal support is highly enzyme-specific and may involve a complex mixture of multiple components, a significant bottleneck in the discovery of optimized copolymer brush support formulations stems from the challenge in rapidly synthesizing and evaluating supports that comprise a large multi-component chemical space. High-throughput approaches have proven useful in the discovery of polymer materials for several applications, and the ability to screen extensive copolymer libraries for optimized enzyme immobilization would potentially enable protein engineers to optimally stabilize enzymes on demand. Previous approaches to find optimal support compositions used conventional low-throughput, empirical approaches to explore different compositions of copolymer brush mixtures, which made the exploration of chemical space laborious and inefficient. For these reasons, the development of a high-throughput approach for the discovery of new immobilization supports would offer tremendous advantages. The present invention provides methods and systems to make such high through-put explorations possible as will be evident in the following disclosure.

### SUMMARY OF THE INVENTION

A novel method, termed “Combinatorial High-throughput Enzyme Support Screening” (CHESS), is provided which is specifically designed for the high-throughput, combinatorial synthesis of random copolymer brush supports of controlled composition and in situ screening of immobilized enzyme activity and stability. This approach involves the preparation of multi-component random copolymer brushes in 384-well microplates via surface-initiated atom transfer radical polymerization (ATRP). To enable ATRP, the walls of each well were modified with a novel ATRP initiator, followed by green-light-activated polymerization. After polymerization and enzyme immobilization, the activity and stability of the immobilized enzyme can be quantitatively characterized as a function of brush composition under thermally and chemically denaturing conditions, or a variety of other conditions of interest (e.g., stability of the enzyme over time, in harsh environments, such as elevated or depressed pH, across a temperate spectrum). This approach was comprehensively characterized and validated using Bacillus subtilis Lipase A (LipA) as a model enzyme, and its utility to predict optimal brushes for previously unexplored enzymes was demonstrated using alkaline phosphatase (AlkP). The results underscore the utility of CHESS compared to conventional empirical approaches for identifying optimal copolymer brush compositions as immobilization supports and illustrate the potential of this approach to develop robust enzyme biocatalysts for industrial biotransformations and other applications.

Also provided is a system and related methodology to anchor a polymer or copolymer to a surface such as polypropylene, polystyrene or polycarbonate. A solution of bis[bromo]benzophenone (2BrBP) in toluene can be deposited on the surface, followed by irradiating the surface with UV-light to crosslink the 2BrBP. Then a monomer or mixture of monomers can be added followed by the initiating the polymerization of the monomers to polymer/copolymer brushes. It is contemplated that this method could be used to prepare a well, bead, flat surface, etc. to immobilize an enzyme.

In a first aspect the present invention provides a method to screen multi-component copolymer compositions as supports for enzymes. The method can include the steps of (1) providing a 2BrBP-functionalized multi-well plate with copolymer brushes affixed to the 2BrBP in each well; (2) adding an enzyme to the copolymer brushes of the wells; (3) adding an enzyme substrate to the wells; (4) exposing the plate to a defined set of conditions or chemicals; and (5) measuring the conversion of substrate in each of the plurality of wells responsive to the conditions or chemicals. The multi-well plate has a plurality of wells and the wells have a defined and unique copolymer brush affixed to the 2BrBP in the well. The copolymer brush is defined in the sense that one knows the relative proportion of the constituent monomers or other components in the brush. The copolymer brush is unique in the sense that there will be other brushes in the plate with a different composition, although the brush may be present in duplicate, triplicate, etc., within the wells of the multi-well plate for the purpose testing repeatability, etc. The copolymer brush in each well is created by systematically varying the relative concentration of the constituent monomers or other constituents used to create the copolymer brush. In this manner the performance of the protein of interest can then be assayed in the unique polymer brush environment of the well.

In an advantageous embodiment, the copolymer brush is formed from an aromatic monomer in combination with a non-aromatic monomer. The co-polymer brush is adapted to stabilize an enzyme to be linked thereto. The aromatic monomer can be ethylene glycol phenyl ether methacrylate (EGPMA), phenyl methacrylate, benzyl methacrylate, diethylene glycol phenyl ether methacrylate, triethylene glycol phenyl ether methacrylate, tetraethylene glycol phenyl ether methacrylate, pentaethylene glycol phenyl ether methacrylate, poly(ethylene glycol) phenyl ether methacrylate, 1-Naphthyl methacrylate, 2-Naphthyl methacrylate, 1-Pyrenemethyl methacrylate, 9-Anthracenemethyl methacrylate, styrene, ethylene glycol phenyl ether acrylate, phenyl acrylate, benzyl acrylate, diethylene glycol phenyl ether acrylate, triethylene glycol phenyl ether acrylate, tetraethylene glycol phenyl ether acrylate, pentaethylene glycol phenyl ether acrylate, poly(ethylene glycol) phenyl ether acrylate, 1-Naphthyl acrylate, 2-Naphthyl acrylate, 1-Pyrenemethyl acrylate, 9-Anthracenemethyl acrylate, ethylene glycol phenyl ether acrylamide, phenyl acrylamide, benzyl acrylamide, diethylene glycol phenyl ether acrylamide, triethylene glycol phenyl ether acrylamide, tetraethylene glycol phenyl ether acrylamide, pentaethylene glycol phenyl ether acrylamide, poly(ethylene glycol) phenyl ether acrylamide, 1-Naphthyl acrylamide, 2-Naphthyl acrylamide, 1-Pyrenemethyl acrylamide and 9-Anthracenemethyl acrylamide, ethylene glycol phenyl ether methacrylamide, phenyl methacrylamide, benzyl methacrylamide, diethylene glycol phenyl ether methacrylamide, triethylene glycol phenyl ether methacrylamide, tetraethylene glycol phenyl ether methacrylamide, pentaethylene glycol phenyl ether methacrylamide, poly(ethylene glycol) phenyl ether methacrylamide, 1-Naphthyl methacrylamide, 2-Naphthyl methacrylamide, 1-Pyrenemethyl methacrylamide and 9-Anthracenemethyl methacrylamide.

The non-aromatic monomer can be [3-(methacryloylamino)propyl]trimethylammonium chloride, [2-(methacryloyloxy)ethyl)]trimethylammonium chloride, 3-sulfopropyl methacrylate, 3-sulfopropyl acrylate, 3-sulfopropyl acrylamide, 3-sulfopropyl methacrylamide, [2-(methacryloyloxy)ethyl]dimethyl-(3-sulfopropyl)ammonium hydroxide, methacrylic acid, acrylic acid, acrylamide, methacrylamide, poly(ethylene glycol) methacrylate, poly(ethylene glycol) acrylate, poly(ethylene glycol) acrylamide, poly(ethylene glycol) methacrylamide, 2-(diethylamino)ethyl methacrylate, 2-(diethylamino)ethyl acrylate, 2-(diethylamino)ethyl acrylamide, 2-(diethylamino)ethyl methacrylamide, n-isopropylacrylamide, 2-N-morpholinoethyl methacrylate, 2-N-morpholinoethyl acrylate, 2-N-morpholinoethyl acrylamide, and 2-N-morpholinoethyl methacrylamide. In a particularly advantageous embodiment the non-aromatic monomer is sulfobetaine methacrylate (SBMA), Phosphorylcholine methacrylate (PCMA), Glycosyloxyethyl methacrylate (GEMA), poly(ethylene glycol) methacrylate (PEGMA).

In a particularly advantageous embodiment the non-aromatic monomer is sulfobetaine methacrylate (SBMA), Phosphorylcholine methacrylate (PCMA), Glycosyloxyethyl methacrylate (GEMA), poly(ethylene glycol) methacrylate (PEGMA).

The non-aromatic monomer can also be a zwitterionic monomer. Zwitterionic monomers include sulfobetaine acrylate, sulfobetaine acrylamide, sulfobetaine methacrylamide, phosphorylcholine acrylate, phosphorylcholine acrylamide, phosphorylcholine methacrylamide, acryloyl serine, acryloyl ornithine, acryloyl lysine, and acryloyl glutamate.

The conversion of substrate can be measured with colorimetric or fluorometric assays.

Enzymes used in the method can be from the groups lipase, carbonic anhydrase, cytochrome P450, benzaldehyde lyase, alkaline phosphatase, trypsin, chymotrypsin, thrombin, subtilisin, horseradish peroxidase, acetylcholinesterase, glucose isomerase, penicillin g acylase, epimerase, phytase, protein A, transaminase, nitroreductase, unspecific peroxygenase, and imine reductase. Similarly, the enzyme can be from an enzyme group selected from the group consisting of oxidoreductases, transferases, hydrolases, lyases, isomerases, ligases, and translocases.

More specifically, the enzyme can Candida rugosa lipase (CRL), Candida antarctica lipase B (CALB), Rhizomucor miehei lipase (RML), Bacillus subtilis lipase A (LipA), Pseudomonas stutzeri triacylglycerol lipase (lipase TL), and lipase from Sphingomonas sp. (HXN-200).

In a second aspect the present invention provides a method of preparing a polymer-functionalized welled plate for enzyme immobilization. The method includes the steps of: (1) depositing a solution of bis[bromo]benzophenone (2BrBP) in toluene into the wells of the plate, (2) irradiating the plate with UV-light to crosslink the 2BrBP, (3) depositing a mixture of monomers in the wells of the plate, and (4) initiating the polymerization of copolymer brushes within the wells using green light. The toluene causes the well material to swell which allows the 2BrBP to intercalate into the walls of the plate. The mixture of monomers in a plurality of the wells systematically varies in relative proportion across a series of the wells. The variation allows for testing to determine an optimized relative mixture of the monomers of other components under controlled conditions. It is contemplated that the wells of the plate are made from polypropylene, polystyrene or polycarbonate.

The copolymer brush can be formed from an aromatic monomer in combination with a non-aromatic monomer. The co-polymer brush is adapted to stabilize an enzyme to be linked thereto.

Aromatic monomers used in certain embodiments can include ethylene glycol phenyl ether methacrylate (EGPMA), phenyl methacrylate, benzyl methacrylate, diethylene glycol phenyl ether methacrylate, triethylene glycol phenyl ether methacrylate, tetraethylene glycol phenyl ether methacrylate, pentaethylene glycol phenyl ether methacrylate, poly(ethylene glycol) phenyl ether methacrylate, 1-Naphthyl methacrylate, 2-Naphthyl methacrylate, 1-Pyrenemethyl methacrylate, 9-Anthracenemethyl methacrylate, styrene, ethylene glycol phenyl ether acrylate, phenyl acrylate, benzyl acrylate, diethylene glycol phenyl ether acrylate, triethylene glycol phenyl ether acrylate, tetraethylene glycol phenyl ether acrylate, pentaethylene glycol phenyl ether acrylate, poly(ethylene glycol) phenyl ether acrylate, 1-Naphthyl acrylate, 2-Naphthyl acrylate, I-Pyrenemethyl acrylate, 9-Anthracenemethyl acrylate, ethylene glycol phenyl ether acrylamide, phenyl acrylamide, benzyl acrylamide, diethylene glycol phenyl ether acrylamide, triethylene glycol phenyl ether acrylamide, tetraethylene glycol phenyl ether acrylamide, pentaethylene glycol phenyl ether acrylamide, poly(ethylene glycol) phenyl ether acrylamide, 1-Naphthyl acrylamide, 2-Naphthyl acrylamide, 1-Pyrenemethyl acrylamide and 9-Anthracenemethyl acrylamide, ethylene glycol phenyl ether methacrylamide, phenyl methacrylamide, benzyl methacrylamide, diethylene glycol phenyl ether methacrylamide, triethylene glycol phenyl ether methacrylamide, tetraethylene glycol phenyl ether methacrylamide, pentaethylene glycol phenyl ether methacrylamide, poly(ethylene glycol) phenyl ether methacrylamide, 1-Naphthyl methacrylamide, 2-Naphthyl methacrylamide, 1-Pyrenemethyl methacrylamide and 9-Anthracenemethyl methacrylamide.

Non-aromatic monomers used in certain embodiments can include sulfobetaine methacrylate (SBMA), Phosphorylcholine methacrylate (PCMA), Glycosyloxyethyl methacrylate (GEMA), poly(ethylene glycol) methacrylate (PEGMA).

More generally, non-aromatic monomers used in certain embodiments can include [3-(methacryloylamino)propyl]trimethylammonium chloride, [2-(methacryloyloxy)ethyl)]trimethylammonium chloride, 3-sulfopropyl methacrylate, 3-sulfopropyl acrylate, 3-sulfopropyl acrylamide, 3-sulfopropyl methacrylamide, [2-(methacryloyloxy)ethyl]dimethyl-(3-sulfopropyl)ammonium hydroxide, methacrylic acid, acrylic acid, acrylamide, methacrylamide, poly(ethylene glycol) methacrylate, poly(ethylene glycol) acrylate, poly(ethylene glycol) acrylamide, poly(ethylene glycol) methacrylamide, 2-(diethylamino)ethyl methacrylate, 2-(diethylamino)ethyl acrylate, 2-(diethylamino)ethyl acrylamide, 2-(diethylamino)ethyl methacrylamide, n-isopropylacrylamide, 2-N-morpholinoethyl methacrylate, 2-N-morpholinoethyl acrylate, 2-N-morpholinoethyl acrylamide, and 2-N-morpholinoethyl methacrylamide.

Similarly, the non-aromatic monomer can be a zwitterionic monomer, such as sulfobetaine acrylate, sulfobetaine acrylamide, sulfobetaine methacrylamide, phosphorylcholine acrylate, phosphorylcholine acrylamide, phosphorylcholine methacrylamide, acryloyl serine, acryloyl ornithine, acryloyl lysine, and acryloyl glutamate.

The method of the second aspect can include the step of depositing an enzyme into the wells after copolymer brush polymerization, whereby the enzyme attaches to the copolymer brush or is otherwise immobilized on the copolymer brush. Deposited enzymes can include lipases, carbonic anhydrase, cytochrome P450, benzaldehyde lyase, alkaline phosphatase, trypsin, chymotrypsin, thrombin, subtilisin, horseradish peroxidase, acetylcholinesterase, glucose isomerase, penicillin g acylase, epimerase, phytase, protein A, transaminase, nitroreductase, unspecific peroxygenase, and imine reductase. In an advantageous embodiment the enzyme is Candida rugosa lipase (CRL), Candida antarctica lipase B (CALB), Rhizomucor miehei lipase (RML), Bacillus subtilis lipase A (LipA), Pseudomonas stutzeri triacylglycerol lipase (lipase TL), and lipase from Sphingomonas sp. (HXN-200). Broadly speaking, the enzyme can be from an enzyme group such as oxidoreductases, transferases, hydrolases, lyases, isomerases, ligases, and translocases.

In a third aspect the present invention provides a high-throughput platform that enables rapid screening of complex and heterogeneous copolymer brushes as enzyme immobilization supports using a multi-well-plate format. The platform can utilize a multi-well plate having arrays of three-component photo-activated surface-initiated polymerization polymer brushes in the wells in combination with immobilized enzymes, whereby the platform facilitates identify optimized immobilization supports under thermally and chemically denaturing conditions.

The platform of the third aspect can be used in a method of screening for polymer brush optimization for an enzyme. The method can be used to predict the optimal composition of polymer brushes a priori for a previously unexplored enzyme.

In a fourth aspect the present invention provides a method of preparing a polymer-functionalized polypropylene surface. The method according to the fourth aspect can include the steps of: (1) dissolving 2BrBP in toluene at an about 10 mM concentration while solution is protected from light; (2) adding the dissolved 2BrBP solution to wells of a PP plate; (3) incubating the plate for about 30 minutes to about 12 hrs (preferably about 2 hrs) in a confined environment, whereby the environment is adapted to avoid solvent evaporation; (4) partially submerging the plate in a bath sonicator wherein the bath liquid is exclusively in contact with the plate skirt and bottom and without removing the 2BrBP solution from the well; (5) sonicating the plate for about 5 to about 60 seconds (preferably about 10 seconds) 10 seconds; removing the remaining 2BrBP solution; curing the well under an approximately 50 W 365 nm UV light source for about 5 minutes to about 1 hr (preferably about 5-20 minutes, most preferably 10 minutes); and washing the plate with dichloromethane, whereby the dichloromethane removes all non-covalently attached 2BrBP. It is contemplated that the method can be used to prepare a variety of surfaces including polypropylene, polystyrene or polycarbonate and the method need not be limited to surfaces having a well architecture (e.g., they could be particles, beads or flat surfaces).

The method of preparing a polymer-functionalized polypropylene surface according to the fourth aspect can include the step of growing copolymer brushes from the wall of 2BrBP-functionalized well plates using a modified version of an oxygen-insensitive ATRP. The polymer precursor solutions can be prepared and mixed in the appropriate ratios to yield the monomer ratios that are summarized in Table 1, below. In an advantageous embodiment, the method of preparing a polymer-functionalized polypropylene surface according to the fourth aspect can employ a plurality of polymer precursor solutions that are prepared using the same combination of monomers at varying concentrations. The concentration of all different monomers (e.g., SBMA, PEGMA, and GMA) can add up to given mM in all cases, to maintain the total amount of monomer independent of monomer ratios. The plates or particles can be kept in a reactor for polymerization, exposing the plates or particles to 100 W green (520-525 nm) LED sources for about 90 min.

The prepared plates or particles can be kept in a reactor for polymerization, exposing the plates or particles to 100 W green (520-525 nm) LED sources for about 30 to about 120 min (preferably 90 min). The method can further include the steps of washing the plates and particles with water and isopropanol and storing in a desiccator until further use.

In a fifth aspect the present invention provides a method to rapidly screen multi-component random copolymer compositions as supports for enzymes. The method includes the steps of: (1) functionalizing the polypropylene (PP) surface with bis[bromo]benzophenone (2BrBP) by depositing a solution of 2BrBP in toluene on the surface, wherein the toluene caused the PP to swell, allowing 2BrBP to intercalate into the walls of the plate; (2) cross-linking the 2BrBP and PP by exposing the wells to UV irradiation; (3) contacting the crosslinked PP-2BrBP with solutions comprising varying concentrations of a plurality of monomers; (4) forming copolymers by reacting the solutions comprising varying concentrations of a plurality of monomers using a green-light-activated SI-ATRP method; and (5) immobilizing an enzyme on the resulting copolymer brush. The method can further include the step of measuring the relative activity of the enzymes in each well of the plate following the immobilizing step. A higher activity implicates an enhanced support for the copolymer support for the enzyme.

In a sixth aspect the present invention provides a method to immobilize an enzyme to a surface. The method includes the steps of: (1) functionalizing the polypropylene, polystyrene or polycarbonate surface with bis[bromo]benzophenone (2BrBP) by depositing a solution of 2BrBP in toluene on the surface, wherein the toluene causes the surface to swell, allowing 2BrBP to intercalate into the surface; (2) cross-linking the 2BrBP and surface by exposing the surface to UV irradiation; (3) contacting the crosslinked PP-2BrBP with solutions comprising a plurality of monomers; (4) forming copolymers by reacting the solutions comprising varying concentrations of a plurality of monomers using green light (e.g., a green-light-activated SI-ATRP method); and (5) immobilizing an enzyme on the resulting copolymer brush.

In further aspects the present invention provides a novel combinatorial and high-throughput platform that enables rapid screening of complex and heterogeneous copolymer brushes that enhance the performance of proteins interacting with the polymer brush surface wherein the platform employs a system for varying the ratios of monomers or other constituents making up the polymer brush surface as disclosed herein (see e.g., Example 1 and Table 1), contacting the varied resulting copolymer brushes with a protein of interest, and measuring one or more parameters for a protein of interest for each resulting copolymer brush.

The novel combinatorial and high-throughput platform that enables rapid screening of complex and heterogeneous copolymer brushes that enhance the performance of proteins interacting with the polymer brush surface can employ a multi-well plate having wells composed of polypropylene (or other well material such as polystyrene or polycarbonate) cross-linked to 2BrBP, to facilitate growth of the copolymer brushes on the surface of the wells. Each well of the multi-well plate can have a defined and unique copolymer brush that is created by systematically varying the relative concentration of the constituent monomers or other constituents used to create the copolymer brush. The performance of the protein of interest can then be assayed in the unique polymer brush environment of a well.

In certain embodiments the copolymer brush is formed from an aromatic monomer in combination with a non-aromatic monomer, wherein the co-polymer brush is adapted to stabilize an enzyme to be linked thereto. The aromatic monomer can be ethylene glycol phenyl ether methacrylate (EGPMA), phenyl methacrylate, benzyl methacrylate, diethylene glycol phenyl ether methacrylate, triethylene glycol phenyl ether methacrylate, tetraethylene glycol phenyl ether methacrylate, pentaethylene glycol phenyl ether methacrylate, poly(ethylene glycol) phenyl ether methacrylate, 1-Naphthyl methacrylate, 2-Naphthyl methacrylate, 1-Pyrenemethyl methacrylate, 9-Anthracenemethyl methacrylate, styrene, ethylene glycol phenyl ether acrylate, phenyl acrylate, benzyl acrylate, diethylene glycol phenyl ether acrylate, triethylene glycol phenyl ether acrylate, tetraethylene glycol phenyl ether acrylate, pentaethylene glycol phenyl ether acrylate, poly(ethylene glycol) phenyl ether acrylate, 1-Naphthyl acrylate, 2-Naphthyl acrylate, 1-Pyrenemethyl acrylate, 9-Anthracenemethyl acrylate, ethylene glycol phenyl ether acrylamide, phenyl acrylamide, benzyl acrylamide, diethylene glycol phenyl ether acrylamide, triethylene glycol phenyl ether acrylamide, tetraethylene glycol phenyl ether acrylamide, pentaethylene glycol phenyl ether acrylamide, poly(ethylene glycol) phenyl ether acrylamide, I-Naphthyl acrylamide, 2-Naphthyl acrylamide, 1-Pyrenemethyl acrylamide and 9-Anthracenemethyl acrylamide, ethylene glycol phenyl ether methacrylamide, phenyl methacrylamide, benzyl methacrylamide, diethylene glycol phenyl ether methacrylamide, triethylene glycol phenyl ether methacrylamide, tetraethylene glycol phenyl ether methacrylamide, pentaethylene glycol phenyl ether methacrylamide, poly(ethylene glycol) phenyl ether methacrylamide, 1-Naphthyl methacrylamide, 2-Naphthyl methacrylamide, 1-Pyrenemethyl methacrylamide and 9-Anthracenemethyl methacrylamide.

The non-aromatic monomer can be sulfobetaine methacrylate (SBMA), Phosphorylcholine methacrylate (PCMA), Glycosyloxyethyl methacrylate (GEMA), poly(ethylene glycol) methacrylate (PEGMA). Similarly, the non-aromatic monomer can be [3-(methacryloylamino)propyl]trimethylammonium chloride, [2-(methacryloyloxy)ethyl)]trimethylammonium chloride, 3-sulfopropyl methacrylate, 3-sulfopropyl acrylate, 3-sulfopropyl acrylamide, 3-sulfopropyl methacrylamide, [2-(methacryloyloxy)ethyl]dimethyl-(3-sulfopropyl)ammonium hydroxide, methacrylic acid, acrylic acid, acrylamide, methacrylamide, poly(ethylene glycol) methacrylate, poly(ethylene glycol) acrylate, poly(ethylene glycol) acrylamide, poly(ethylene glycol) methacrylamide, 2-(diethylamino)ethyl methacrylate, 2-(diethylamino)ethyl acrylate, 2-(diethylamino)ethyl acrylamide, 2-(diethylamino)ethyl methacrylamide, n-isopropylacrylamide, 2-N-morpholinoethyl methacrylate, 2-N-morpholinoethyl acrylate, 2-N-morpholinoethyl acrylamide, and 2-N-morpholinoethyl methacrylamide.

The non-aromatic monomer can be a zwitterionic monomer, such as sulfobetaine acrylate, sulfobetaine acrylamide, sulfobetaine methacrylamide, phosphorylcholine acrylate, phosphorylcholine acrylamide, phosphorylcholine methacrylamide, acryloyl serine, acryloyl ornithine, acryloyl lysine, and acryloyl glutamate.

In advantageous embodiments the protein of interest is an enzyme attached to the copolymer brush. The enzyme can be a lipase, carbonic anhydrase, cytochrome P450, benzaldehyde lyase, alkaline phosphatase, trypsin, chymotrypsin, thrombin, subtilisin, horseradish peroxidase, acetylcholinesterase, glucose isomerase, penicillin g acylase, epimerase, phytase, protein A, transaminase, nitroreductase, unspecific peroxygenase, and imine reductase. Similarly, the enzyme can be Candida rugosa lipase (CRL), Candida antarctica lipase B (CALB), Rhizomucor miehei lipase (RML), Bacillus subtilis lipase A (LipA), Pseudomonas stutzeri triacylglycerol lipase (lipase TL), and lipase from Sphingomonas sp. (HXN-200). Broadly speaking, the enzyme is from an enzyme group selected from the group consisting of oxidoreductases, transferases, hydrolases, lyases, isomerases, ligases, and translocases.

### BRIEF DESCRIPTION OF THE DRAWINGS

For a fuller understanding of the invention, reference should be made to the following detailed description, taken in connection with the accompanying drawings, in which:

FIG. 1 is an image showing a schematic of 384-well plate functionalization for enzyme immobilization. (A) Structure of bis[bromo]benzophenone (2BrBP) with schematic representation of core UV-crosslinker benzophenone moiety and polymerization initiation moieties. (B) Modification of the walls of the PP plate with 2BrBP via solvent-induced swelling and subsequent crosslinking using UV light. Following crosslinking, green light was used to initiate polymerization of random copolymer brushes that coated the wall of the individual wells. (C) Schematic representation of a well functionalized with polymer brushes serving as immobilization support for an enzyme. Incorporation of glycidyl methacrylate-bearing (GMA) epoxide groups within the brush layer enables covalent immobilization of the enzyme.

FIG. 2 is a drawing showing patterning of 384-well PP plates with 2BrBP. 2BrBP initiator was deposited in wells (thick black circles), forming the shape of a smiley face. Polymerizations containing 2% RhBA were performed in all wells of the plate, and unreacted monomer was washed away with ethanol. RhBA fluorescence in the wells was measured in a plate reader using emission and excitation wavelengths of 548 and 570 nm, respectively. The color scale for fluorescence was normalized to the fluorescence of the well with the highest fluorescent intensity in the plate (shown in grayscale in the figure).

FIG. 3 is a schematic workflow for analysis of LipA activity data. Changes in fluorescence over time were measured in each well using a fluorescent plate reader. Background product formation from wells without enzyme was subtracted from that of the enzyme-containing wells in the same column, and the slopes of the initial rate were calculated. Finally, the four brush composition replicates in 2×2 squares were aggregated by averaging their values and plotted as a heatmap using the conversion between fluorescence units and concentration from a standard curve for the fluorescent product compound.

FIG. 4 is a set of images showing relative activity retention of immobilized LipA at 50° C. on a plate with varying compositions of sulfobetaine methacrylate (SBMA), poly(ethylene glycol) methacrylate (PEGMA), and GMA. (A) Heatmap of LipA activity after immobilization prior to incubation at elevated temperature. (B) Heatmap of LipA activity after incubation for 14 days at 50° C. The top and bottom panels share the same color scale, which is normalized to the range between the highest activity in panel (A) and the lowest activity in panel (B).

FIG. 5 is a set of graphs showing thermal deactivation of immobilized LipA as measured by CHESS and by reactions using biocatalyst particles. (A) Heatmap of tin for immobilized LipA. Selected compositions used for validation by biocatalyst particles are highlighted by squares and labeled with an identifying number. The values represented in the heatmap correspond to the mean tin accounting for data from the three experimental replicates. The color table is scaled from the lowest to highest value of tin in the compositional array. (B) Comparison of relative activity retention profiles and tin values for the selected brush compositions between well-plate and particle data. The profile for each experimental replicate was fit to a first-order exponential decay function, and the annotated tin values correspond to the mean and standard deviation of the individual tin values from each exponential fit. The prediction bands correspond to the 95% confidence interval for exponential fits for the well-plate data, generated using GraphPad Prism software. The error bars for plate data (black) represent the propagated standard error of the mean for the 4 technical replicates in each experimental replicate (n=12). The error bars for particles data (gray) represent the standard error of the mean for 3 technical replicates (n=3).

FIG. 6 is a set of graphs showing a comparison of urea-induced chemical inactivation of LipA in well-plates and on biocatalyst particles. (A) Heatmap of C₅₀ for immobilized LipA in well-plates. Selected compositions used for validation on particles are highlighted and labeled with an identifying number. The values represented in the heatmap correspond to the mean C₅₀ across the experimental trials. The color bar (shown in grayscale) ranges from the lowest to highest value of C₅₀ in the compositional array. (B) Chemical inactivation profiles for each selected composition from the top panel for well-plate and particle data. The profile for each experimental replicate was fit to a variable-slope sigmoidal function, and the annotated C₅₀ values correspond to the mean and standard deviation of the individual C₅₀ values from each sigmoidal fit. The prediction bands correspond to the 95% confidence interval for sigmoidal fit of the well-plate data, generated using GraphPad Prism software. The error bars for well-plate data represent the propagated standard error of the mean for the four technical replicates in each experimental replicate (n=12). The error bars for particle data represent the standard error of the mean for three technical replicates (n=3).

FIG. 7 is a set of graphs showing a statistical analysis comparing CHESS and biocatalyst particle data from thermal and chemical denaturation experiments. (A) Pearson correlation to compare thermal deactivation activities from CHESS and particle data for each brush support composition from FIG. 5. (B) Bland-Altman plot to characterize the bias and reliability of CHESS in thermal denaturation experiments. A positive value of mean difference suggests that the data from CHESS is on average higher in value than data from the particles. (C) Pearson correlation comparing CHESS and particle data from FIG. 6. The value of the Pearson coefficient was calculated with GraphPad Prism. (D) Bland-Altman plot used to characterize bias and reliability of CHESS measurements. A negative value of mean difference suggests that data from CHESS is typically lower in value than data from the particles. GraphPad Prism was used to calculate Pearson coefficients in panels (A) and (C), as well as to determine Bland-Altman plots and parameters.

FIG. 8 is a set of graphs showing the activity retention of immobilized AlkP after incubation at 50° C. for 88 h on a SBMA/PEGMA/GMA plate. (A) Heatmap of relative activity retention. The color scale (shown in grayscale) was normalized from the lowest to the highest values for relative activity retention across the well plate. Seven polymer compositions representing different levels of stabilization and a variety of compositions were selected for detailed comparison with biocatalyst particles (dark square outlines). (B) Bar plot representing paired activities comparing the activity of AlkP immobilized on particles or from CHESS. For each bar, the error bars represent the standard error of the mean for the measurements, with three replicates for data from particles (n=3) and four replicates for CHESS (n=4). For each pair, individual t-tests indicated no statistically significant differences (n.s.; p-values>0.05). The inset plot represents the paired particles and CHESS data, where a Pearson coefficient of r=0.983 revealed exceptional correlation between particles and CHESS measurements. Error bars are the same as in the bar plot.

FIG. 9 is a set of drawings and graphs showing the synthesis and characterization of 2BrBP. (A) Reaction scheme for synthesis of 2BrBP as described in the Methods, below. (B) ¹H and (C)¹³C NMR spectra of 2BrBP, with all assignments for each proton and carbon, respectively. Assignments were performed using Mestrelab-Mnova software, and integration analysis showed the expected 3:2 ratio between the 12 aliphatic protons of the a-bromoisobutyryl moieties and the 8 protons of the aromatic benzophenone rings. In the ¹H spectrum, the CDCl₃ signal was eliminated by the software to avoid interference with integration analysis due to overlapping with the aromatic peaks at d=˜7.3 ppm.

FIG. 10 is a set of graphs showing XPS spectra of nitrogen, sulfur and bromine signals for the three steps of PP functionalization in a polypropylene slide. Nitrogen and sulfur signals correspond to the SBMA monomer, and the bromine signal corresponds to the 2BrBP initiator.

FIG. 11 is a graph showing estimated dry thickness for polymer brushes for different SBMA-to-PEGMA ratios. Polymerization conversion was estimated using ¹H-NMR by measuring the disappearance of methacrylate peak (δ=6.08 ppm), estimating the dry volume of polymer and dividing by the surface area of each well. Error bars correspond to the standard error of the mean for three technical replicates (n=3).

FIG. 12 is a pair of graphs showing the characterization of inter-well fluorescence cross-talk. (A) Fluorescence heatmap of a plate where 10 μM of 4-methylumbelliferone in 50 mM sodium phosphate buffer at pH 7 was seeded in four arbitrary wells. All other wells contained exclusively buffer. (B) Relative fluorescence of wells contiguous to seed wells. The contiguous wells used for this analysis are represented in light gray boxes in (A). The relative fluorescence from one well to an adjacent well was estimated as approximately 0.3%. Error bars correspond to the standard deviation for 16 replicates (n=16).

FIG. 13 is a heatmap showing an estimation of LipA loading for each well composition. Each square corresponds to the mean of the four wells in a 2×2 square of the same brush composition.

FIG. 14 is a heatmap showing activity retention for immobilized LipA after incubation at 50° C. for 14 days. For each composition, values are calculated by dividing the activity at t=14 days (FIG. 4(B)) by the initial activity (FIG. 4(A)). The color/grayscale bar is scaled from the lowest to the highest value of activity retention across the whole plate.

FIG. 15 is a set of graphs showing activity retention plots from FIG. 4 with visualization of measurement uncertainty. (A) 3D bar plot with height corresponding to rate of reaction at t=0 days from FIG. 4a. (B) 3D bar plot with height corresponding to rate of reaction at t=14 days from FIG. 4b. (C) Ranked activities from panel (A) and (B). In all panels, error bars correspond to the propagated standard deviation of the mean across quadruplicates of all three experimental replicates (n=12).

FIG. 16 is a graph showing urea-induced chemical inactivation curve for LipA. Measurements were taken after 3 h equilibration of enzyme in the target urea concentration prior to addition of substrate in all buffers containing urea. C₅₀ was determined from the sigmoidal fit to the activity data. Error bars for standard error of the mean for three replicates (n=3) are smaller than the size of the symbols.

FIG. 17 is a schematic showing high-throughput screening of optimal enzyme-copolymer pairs.

FIG. 18 is a graph showing an enzymatic reaction occurring on the walls of well plate. No enzyme was immobilized in the top four rows (A-D). Horseradish peroxidase (HRP) was immobilized in rows E-P. Upon addition of the HRP substrates, hydrogen peroxide and Amplex Red, to all the wells of the well plate, pink color quickly appeared localized to the walls of the wells where HRP had been immobilized. Substrate was pipetted from row A to row P, and the picture was taken 2 min after addition of substrate to row P.

FIG. 19 is an image showing a representative array where thermal deactivation profiles for each quadruplicate is fit to a first-order exponential decay function. The time constant from each fit is used to calculate t_{1/2} for each polymer composition.

FIG. 20 is an image showing a representative array where chemical deactivation profiles for each quadruplicate are fit to a variable-slope sigmoidal function, and the half deactivation constant (C₅₀) is extracted for each of these fits.

### DETAILED DESCRIPTION OF THE PREFERRED EMBODIMENT

Recent advances have demonstrated the promise of complex multi-component polymeric supports to enable supra-biological enzyme performance. However, the discovery of such supports has been limited by time-consuming, low-throughput synthesis and screening. Here, we describe a novel combinatorial and high-throughput platform that enables rapid screening of complex and heterogeneous copolymer brushes as enzyme immobilization supports named Combinatorial High-throughput Enzyme Support Screening (CHESS). Using a 384 well-plate format, we synthesized arrays of three-component polymer brushes in the microwells using photo-activated surface-initiated polymerization, and immobilized enzymes in situ. The utility of CHESS to identify optimal immobilization supports under thermally and chemically denaturing conditions was demonstrated using Bacillus subtilis Lipase A (LipA). The identification of supports with optimal compositions was validated by immobilizing LipA on polymer-brush modified biocatalyst particles. We further demonstrated that CHESS could be used to predict the optimal composition of polymer brushes a priori for the previously unexplored enzyme, alkaline phosphatase (AlkP). Our findings demonstrate that CHESS represents a predictable and reliable platform for dramatically accelerating the search of chemical compositions for immobilization supports and further facilitate the discovery of biocompatible and stabilizing materials.

Typically, generating one composition of support is a rather laborious process, which takes approximately five days. With the method taught herein, one can generate 384 different compositions at once and immobilize enzymes in each of the compositions in approximately 2 days. This represents a ˜1,000-fold faster throughput than previous conventional methods. In addition, the activity of all enzymes immobilized in the 384 wells can be easily and simultaneously measured with colorimetric or fluorometric assays.

We describe the development of a novel molecule 2BrBP to enable polymerization on polypropylene material (the material that 384 well plates are made of). For context, polypropylene is a very inert material which is quite hard to functionalize. The present invention helps to overcome this difficulty.

The present invention provides ability to screen large variants of surface-grafted copolymers made of complex mixtures of different monomers. The systems and methods taught herein will find particular applicability in the field of chemical/pharmaceutical manufacturing that uses enzyme catalysis, but this approach could be extended to a wide set of other applications including soluble mineral capture, antimicrobial surfaces and others.

It is further envisioned that the system and methods taught herein can be used to identify materials (e.g., polymer brushes) that preserve protein structure and prevent unfolding of proteins (e.g., serum proteins) for tissue engineering and biomaterial applications. In that manner, polymer brushes having optimized structures can be utilized as surface coatings on a wide variety of biologically important surfaces other similar environments where enzyme or protein stability is sought.

While we have used it for immobilization of enzymes on polymers, it can be used to screen multiple surface-grafted polymers, among numerous other applications as will be evident in the disclosure that follows.

### Example 1—Materials and Methods

Materials: Copper (II) bromide, 4,4′-dihydroxybenzophenone, a-bromoisobutyryl bromide, triethylamine, Horseradish Peroxidase, Greiner tall 384 well plates, 4-methylumbelliferyl phosphate, 4-methylumbelliferyl butyrate and 4-methylumbelliferone were purchased from Millipore Sigma (St. Louis, MO). Dichloromethane, methanol, toluene, 2-propanol, hydrogen peroxide and dimethyl sulfoxide were purchased from Fisher Scientific (Waltham, MA). Eosin Y was purchased from MP Biomedicals (Cleveland, OH). Tris[(4-dimethylaminiopyridyl)methyl]amine (TPMA) was purchased from AmBeed (Arlington Hts, IL). Acryloxyethyl thiocarbamoyl Rhodamine B (RhBA) was purchased from Polysciences (Warrington, PA). Amplex Red was purchased from VWR (Radnor, PA). Polypropylene particles (2.45 mm diameter) were obtained from Cospheric (Santa Barbara, CA). Bacillus subtilis LipA was expressed in Escherichia coli, and bovine alkaline phosphatase from intestinal mucosa was obtained from Millipore Sigma (St. Louis, MO).

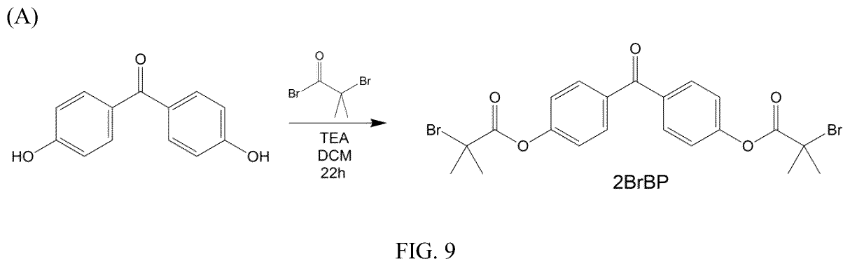

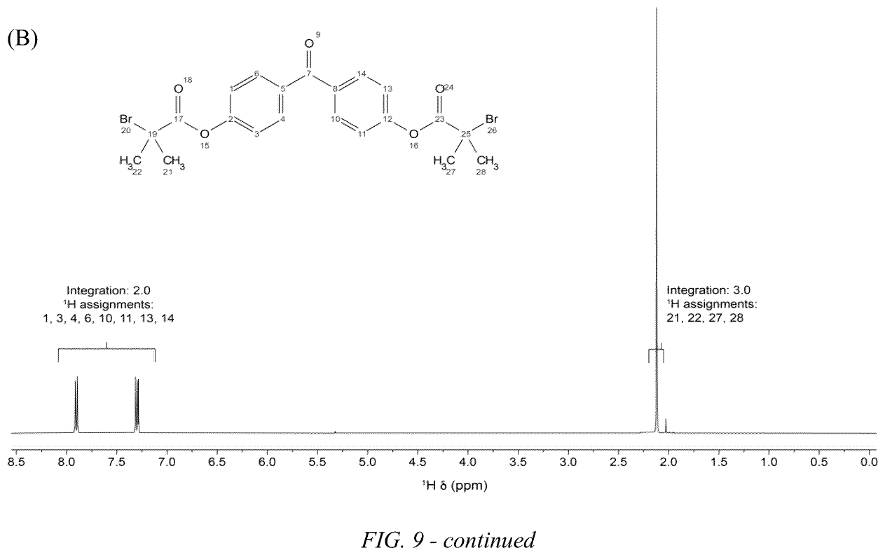

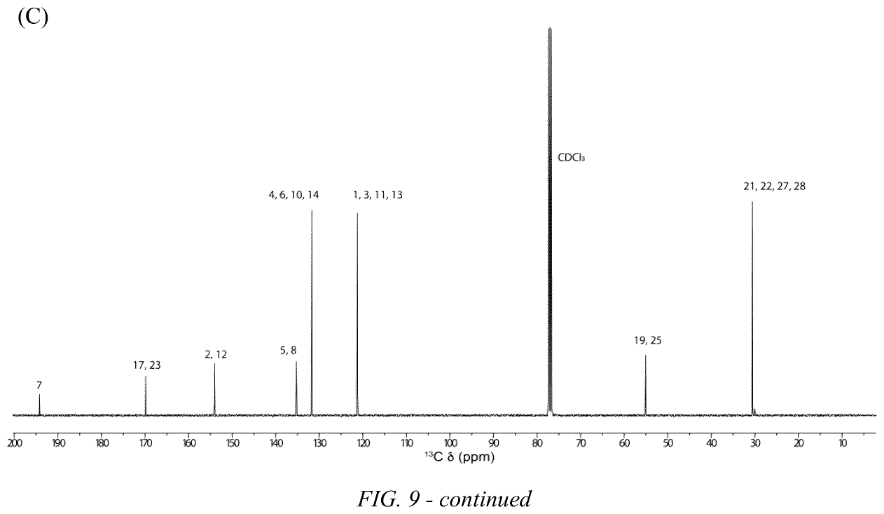

2BrBP Synthesis: The synthesis and characterization of 2BrBP is summarized in FIG. 9, where 4,4′-dihydroxybenzophenone reacts with α-bromoisobutyryl bromide via amine-catalyzed esterification in anhydrous conditions protected from light. For this reaction, 15 g of 4,4-dihydroxybenzophenone was dissolved in 80 mL of anhydrous dichloromethane and 21.35 mL of anhydrous triethylamine were added to this solution, which was then cooled down to 0° C. In parallel, 19.04 mL of alpha-bromoisobutyryl bromide (BIBB) was mixed with 20 mL of anhydrous dichloromethane, and added dropwise to the mixture over 30 min. After 2 h, the reaction was moved to room temperature and allowed to continue for another 20 h. Subsequently, the mixture was filtered through a cellulose filter and the resulting liquid was transferred to an extraction funnel followed by repeated washing (3 times) with ultrapure water and a single wash with a solution of saturated sodium chloride in water. After each wash step, the aqueous phase was discarded and only the organic phase was kept. Finally, the remaining organic phase was dried with anhydrous magnesium sulfate, filtered in vacuo through a cellulose filter, and the solvent was evaporated using a rotary evaporator, yielding a final reaction yield of 57.1%. For characterization, NMR spectra were acquired using a 400 MHz Bruker ADVANCE Spectrometer, and structure analysis and peak integration was performed using Mestrelab-Mnova software. ¹H NMR (400 MHz, CDCl₃) δ 7.88-7.92 (m, 4H), 7.28-7.32 (m, 4H), 2.12 (s, 12H).

### Well Plate Functionalization and Enzyme Immobilization:

Prior to 384-well plate functionalization, 2BrBP was dissolved in toluene at a 10 mM concentration protected from light. Following dissolution, 2BrBP solution was added to all wells and incubated for 2 h with a lid to avoid solvent evaporation. After incubation, the plate was partially submerged in a bath sonicator (i.e., with water exclusively in contact with the plate skirt and bottom), without removing the 2BrBP solution, and sonicated for 10 s. Immediately afterwards, all remaining 2BrBP solution was removed, and the plate cured under a 50 W 365 nm UV light source for 10 min. The plate was then washed extensively with dichloromethane to remove all non-covalently attached 2BrBP. The initiator-functionalized plates were stored in a desiccator until further use.

Random copolymer brushes were grown from the wall of 2BrBP-functionalized well plates using a modified version of an oxygen-insensitive ATRP. Polymer precursor solutions were prepared, and mixed in the appropriate ratios to yield the monomer ratios that are summarized in Table 1. These reactions were carried out using a mixed solvent composed of 80% aqueous 50 mM sodium phosphate buffer at pH 7, 10% DMSO and 10% 2-propanol. In this mixed solvent, reagents for ATRP were dissolved prior to addition of monomers. In all reactions, the final concentrations were: [Eosin Y]=15 μM, [CuBr₂]=0.3 mM, [TPMA]=0.9 mM, [monomers]=180 mM. The concentration of all different monomers (SBMA, PEGMA, and GMA) added up to 180 mM in all cases, to maintain the total amount of monomer independent of monomer ratios. The plates or particles were kept in reactor for polymerization, exposing the plates or particles to two 100 W green (520-525 nm) LED sources for 90 min. After reaction completion, plates and particles were washed thoroughly with water and isopropanol and stored in a desiccator until further use.

Enzyme was immobilized on plates or particles by exchanging enzyme buffer into 10 mM bicine buffer containing 100 mM sodium chloride at pH 8.7, and allowing enzyme attach to brushes during incubation for 12 h at 4° C. (for immobilization in plates), or in a rotating tube that contained enzyme solution and polymer-functionalized particles for 12 h at 4° C. (for immobilization to particles). After immobilization, all non-covalently attached enzyme was removed by washing multiple times with a high ionic strength buffer (50 mM sodium phosphate, pH 7, 500 mM sodium chloride).

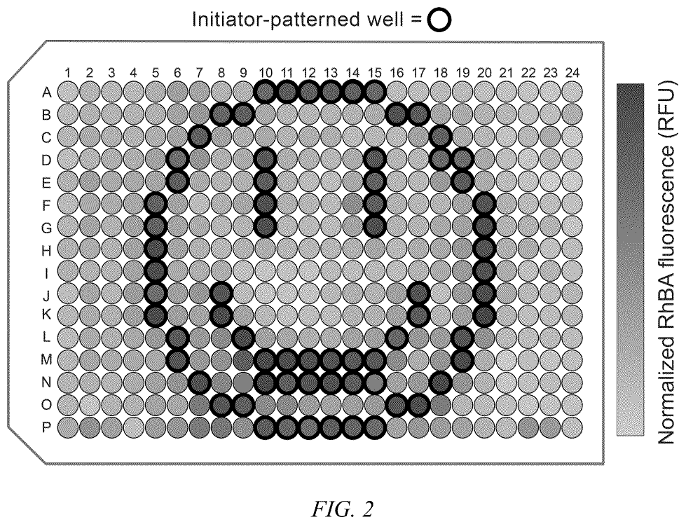

For modification of plates in defined patterns, initiator was deposited exclusively in wells that corresponded to the pixelated smiley-face image in FIG. 2. After applying the steps for initiator functionalization as described above, green light activated polymerizations were performed using 98:2 molar PEGMA:RhBA monomer solution, which was added to all wells of the 384 well plate. Finally, fluorescence mapping was recorded using a SpectraMax iD3 well plate reader using excitation and emission wavelengths of 548 nm and 570 nm, respectively.

### Activity Assays:

LipA activity was assayed using 50 mM sodium phosphate buffer at pH 7 in all cases, and 4-methylumbelliferyl butyrate was used as substrate at a final concentration of 100 μM. For all activity assays, the signal associated with the background substrate hydrolysis reaction was subtracted from kinetic measurements to obtain the true initial catalysis rates. Activity was measured by monitoring the release of fluorescent product and converting fluorescence units to concentration of released product using a standard calibration curve for the product (4-methylumbelliferone), using 365 nm and 465 nm as excitation and emission wavelengths, respectively. Between different activity measurements, buffer containing substrate and reacted product was removed and fresh buffer was added for incubation. For activity assays in well-plate experiments, fluorescent product release was monitored using a SpectraMax iD3 plate reader with temperature controlled at 25° C. For activity measurements using biocatalyst particles, the particles were added to buffer in a temperature-controlled cuvette of a Horiba Scientific Fluoromax-4 spectrofluorometer, and the fluorescent intensity of released product was monitored following equilibration of particle-bound enzyme in buffer.

For well-plate thermal denaturation measurements, we exchanged the buffer used for incubation with new fresh buffer, allowed an incubation period of 20 min at room temperature and added substrate to measure activity. Following acquisition of activity, fresh buffer was replenished in all wells and incubated again in a convection oven at the target temperature (50° C. in this study) until the next measurement. In each incubation step, plates were sealed with a polyester film to prevent evaporation. In chemical denaturation experiments, the corresponding concentration of urea (0-8 M) was added while keeping sodium phosphate at 50 mM and pH at 7, respectively. Additionally, for each increasing step of urea concentration, immobilized enzyme was incubated for 3 h with the target urea concentration prior to assaying activity. For activity assays with AlkP, a final substrate concentration of 100 μM 4-methylumbelliferyl phosphate was used in buffer consisting of 20 mM glycylglycine at pH 8.5 supplemented with 50 μM MgSO₄ and 100 μM ZnSO₄.

### Statistical Analysis:

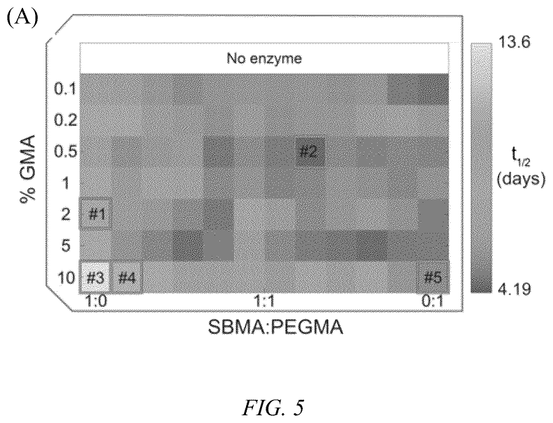

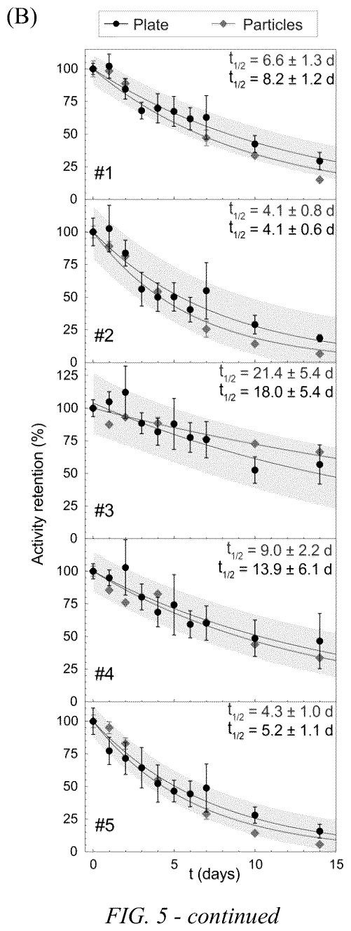

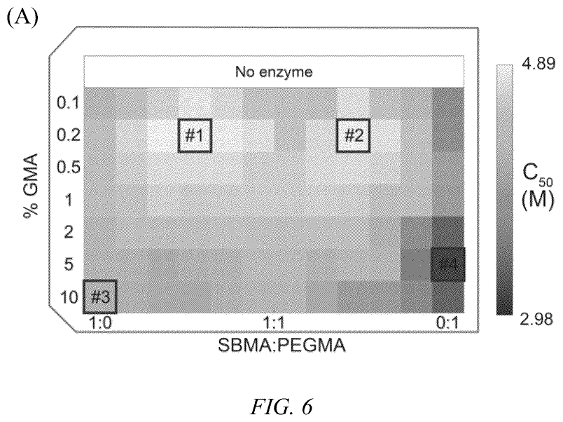

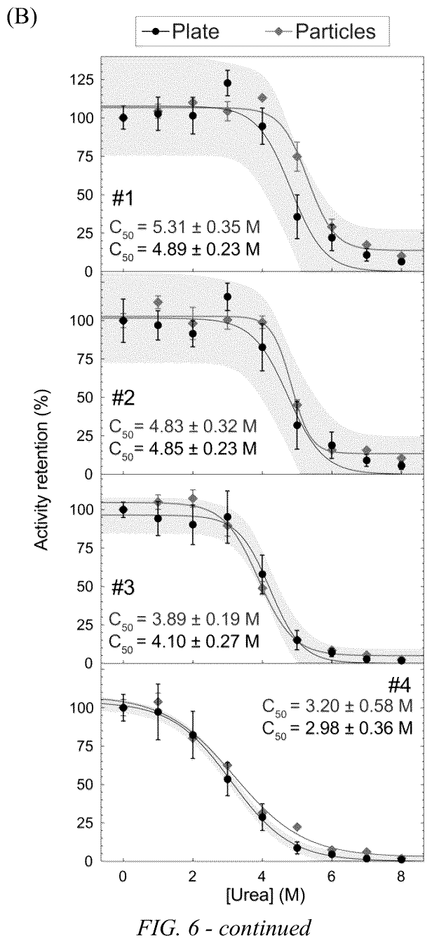

Statistical analyses for the fits in FIGS. 5(B) and 6(B) were generated using GraphPad Prism 10.0.3. For thermal deactivation experiments, biocatalyst particle and well-plate data were fit to first-order exponential decay functions, from which the half deactivation times (t_{1/2}) were calculated, and the 95% prediction bands for the fit of the plate data were generated by the software. For the chemical inactivation experiments, the enzyme inactivation data were fit to a two-state variable response sigmoidal curve, and the half inactivation concentration (C₅₀) was extracted from the software fit. Similarly, the 95% prediction bands of the sigmoidal fit for the plate data were generated by the software.

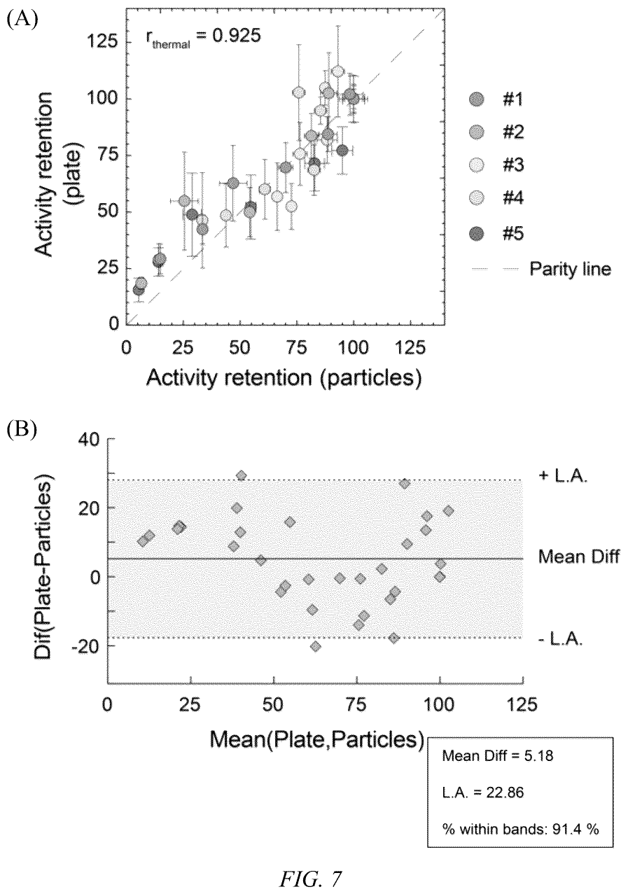

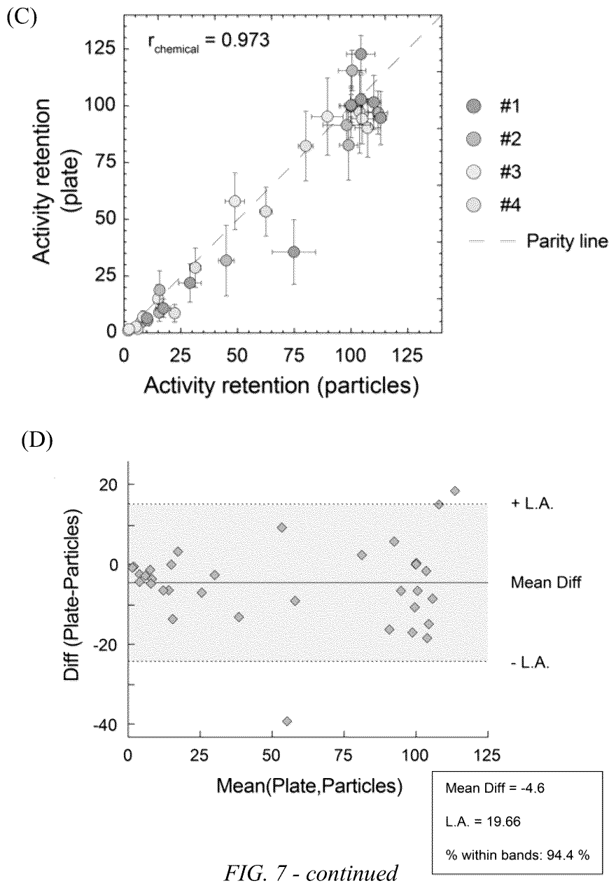

The correlation and potential bias between the well-plate and biocatalyst particle measurements in FIGS. 5(B) and 6(B) were assessed using GraphPad Prism as well via the Pearson coefficient and Bland-Altman differences plot as summarized in FIG. 7. In the Bland-Altman plot, the mean of all paired activity retention data points (x-axis) were compared to the differences of plate minus particle activity retention measurements (y-axis). The plot was centered around the mean difference between plate minus particle data points, and the limits of agreement (L.A.) were calculated as:

$$L . A . = 1.96 \times SD [ Diffs ( Plate - Particles ) ]$$

where SD[Diffs(Plate−Particles)] corresponded to the standard deviation of the differences between well plate-measured activity retention and biocatalyst particle-measured activity retention points for a given incubation time (thermal denaturation experiments) or urea concentration (chemical denaturation experiments). After plotting the mean difference and limits of agreement in the Bland-Altman plot, the percentage of acceptable agreement (%_{AA}) between the two methods across the range of values of activity retention was measured as:

$${\%}_{AA} = \frac{\# data points within L . A . bands}{\# total data points} \times 100$$

To quantify the reliability in comparing measurements between plate and particles, we determined the Intraclass Correlation Coefficient (ICC) for a one-way random effects model as follows:

$$ICC = \frac{\overline{{σ}_{pair}^{2}} - \overline{{σ}_{method}^{2}}}{\overline{{σ}_{pair}^{2}} + ( k - 1 ) \cdot \overline{{σ}_{method}^{2}}}$$

where σ̅ₚ̅ₐ̅ᵢ̅ᵣ̅²̅ corresponds to the mean variance within paired data for both methods, and σ̅_̅{̅m̅e̅t̅h̅o̅d̅}̅²̅ method corresponds to the mean variance between the plate and particles methods. ICC denotes good reliability between two methods if 0.9>ICC>0.75, and excellent reliability for ICC>0.9.

### Example 2—Surface Functionalization and Polymerization

We previously identified random copolymer brush supports that improved the performance of diverse immobilized enzyme biocatalysts by adjusting the hydrophilicity/hydrophobicity of the support using mixtures of sulfobetaine methacrylate (SBMA, hydrophilic) and poly(ethylene glycol) methacrylate (PEGMA, hydrophobic). These brushes also contained glycidyl methacrylate (GMA), an epoxide-containing monomer that allowed covalent attachment of enzymes. While modeling enzyme surface hydrophobicity in conjunction with empirical experiments aided in the development of a predictive rule for selecting immobilization supports, this approach in practice was limited to a widely-spaced exploration of the unidimensional SBMA/PEGMA chemical space using a small number of two-component compositions. Consequently, it was limited in its ability to make precise predictions for more complex polymer supports involving additional components.

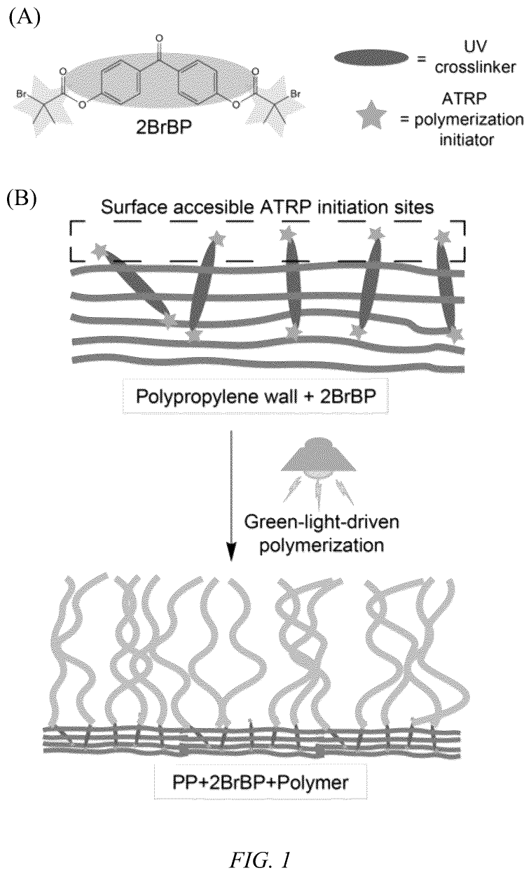

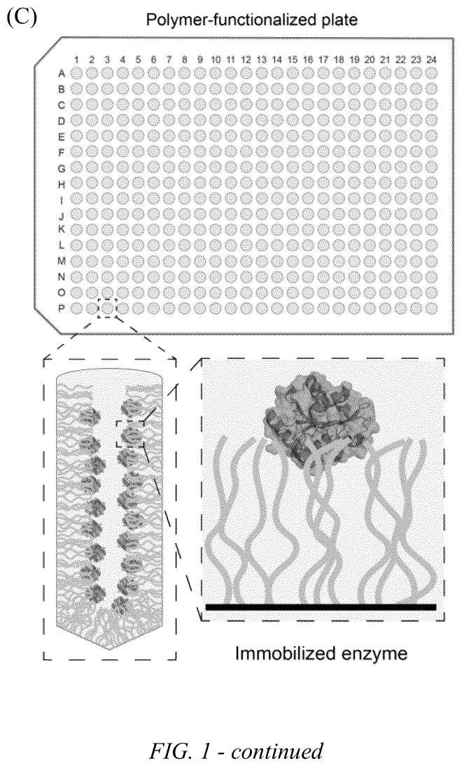

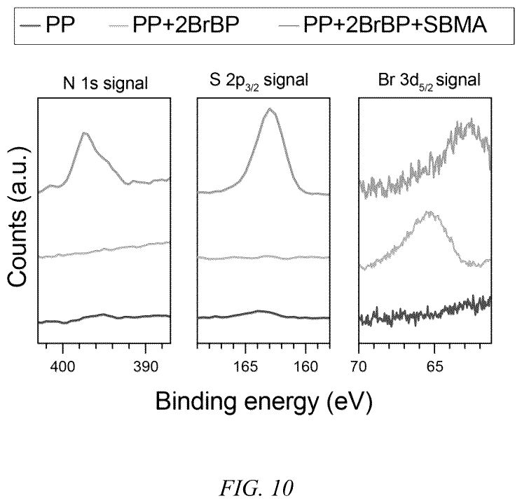

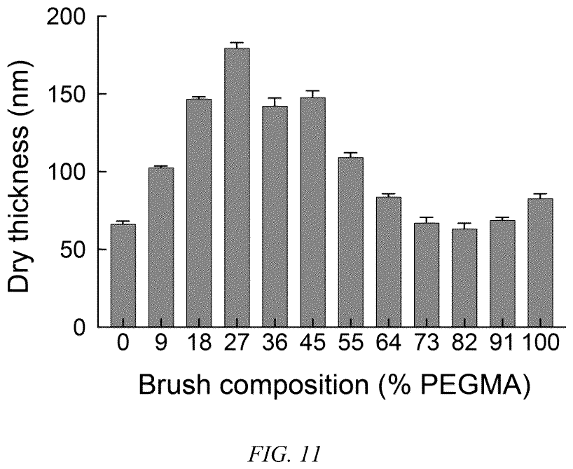

To overcome this practical limitation, we developed a method to rapidly screen multi-component random copolymer compositions as supports for enzymes, which we refer to as CHESS. As part of this approach, the walls of each well in a polypropylene (PP) 384 well-plate were functionalized with the novel ATRP initiator bis[bromo]benzophenone (2BrBP) (FIG. 1(A)), which was synthesized via the reaction of 4,4′-dihydroxybenzophenone with α-bromoisobutyryl bromide (FIG. 9). Importantly, 2BrBP contains tertiary bromides necessary for ATRP initiation, as well as a benzophenone group that can covalently crosslink with PP upon exposure to ultraviolet light. To functionalize the walls of the inert PP well plates, we used solvent-assisted swelling, in which a solution of 2BrBP in toluene was added to the wells. The toluene caused the PP to swell, allowing 2BrBP to intercalate into the walls of the plate. Crosslinks between 2BrBP and PP were subsequently formed via the generation of UV-triggered radicals from the benzophenone moiety. Following crosslinking, random copolymer brushes were grown from the surface of the wells in a custom-made reactor via the oxygen-tolerant green-light-activated SI-ATRP method described by Szczepaniak and coworkers (FIG. 1(B)). The crosslinking of 2BrBP with PP and subsequent polymerization were verified using X-ray photoelectron spectroscopy (FIG. 10). The composition of the polymer brush layer at the walls after polymerization was dependent upon the monomer composition in each well (since the methacrylate monomers have similar reactivity, as verified in previous work), and we determined that the dry thickness of the brushes ranged between 63 to 180 nm (FIG. 11). Finally, incorporation of GMA within the polymer brushes permitted the immobilization of enzymes to the brush layer on the walls of each well (FIG. 1(C)). Notably, epoxides in GMA can react covalently with multiple solvent exposed nucleophilic groups on the surface of enzymes, which may include the N-terminus as well as the side chain of residues such as histidines, lysines, tyrosines and cysteines.

While the present disclosure teaches the use of 2BrBP with polypropylene, it is envisioned that other surfaces typically used for the material in multi-well plates, including polystyrene or polycarbonate, could be modified in the manner taught herein for polypropylene. In other words, 2BrBP could be crosslinked to a material such as polystyrene or polycarbonate. Similarly, while our work was performed with 384 well-plates, there are other formats (e.g., 12, 24, 48, 96, and 1536 well-plates) that are contemplated or other surface geometries to which one might want to mount a polymer brush for screening are contemplated.

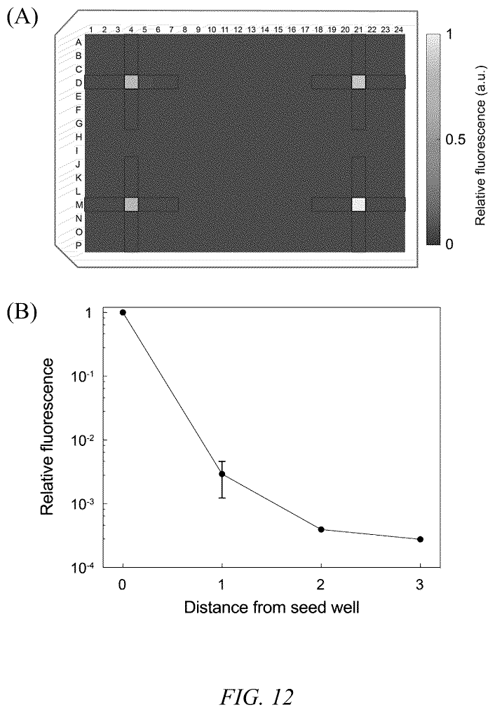

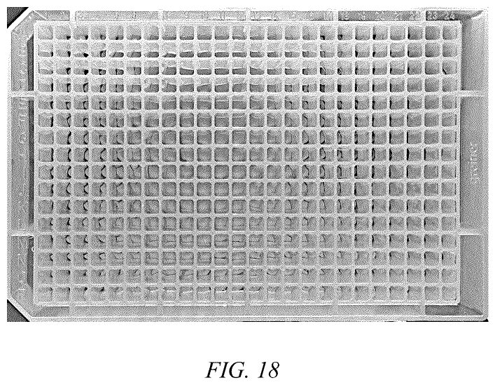

To verify the spatially resolved formation of the brush layer from surface-bound 2BrBP, the wells of a PP microtiter plate were functionalized with 2BrBP in a “smiley-face” pattern, after which the entire plate was filled with a polymerization solution containing a 2:98 molar ratio of the fluorescent monomer Rhodamine B acrylate (RhBA) to PEGMA. Because PEGMA brushes with RhBA should only grow in the wells functionalized with 2BrBP, a fluorescent pattern in the form of the original smiley-face was expected. After polymerization with green light and removing unreacted monomer, an intense fluorescent signal was observed from the wells that had been functionalized with the 2BrBP initiator (FIG. 2). As such, the fluorescent signal yielded a distinct smiley-face pattern, which confirmed that polymerization occurred only when 2BrBP was present in the wells. Moreover, to demonstrate the ability to immobilize enzymes to the brush layer, horseradish peroxidase (HRP) was covalently conjugated to homopolymer brushes composed of GMA. After removing any adsorbed (i.e., non-covalently attached) HRP and adding hydrogen peroxide and Amplex Red, the formation of resorufin from the HRP-catalyzed oxidation of Amplex Red was immediately observed at the walls of the wells (FIG. 18). Additionally, although the microtiter plates were translucent, the bleeding of fluorescent signal into adjacent wells was negligible (FIG. 12).

### Example 3—Measurement and Mapping of Enzymatic Activity in Microtiter Well-Plate

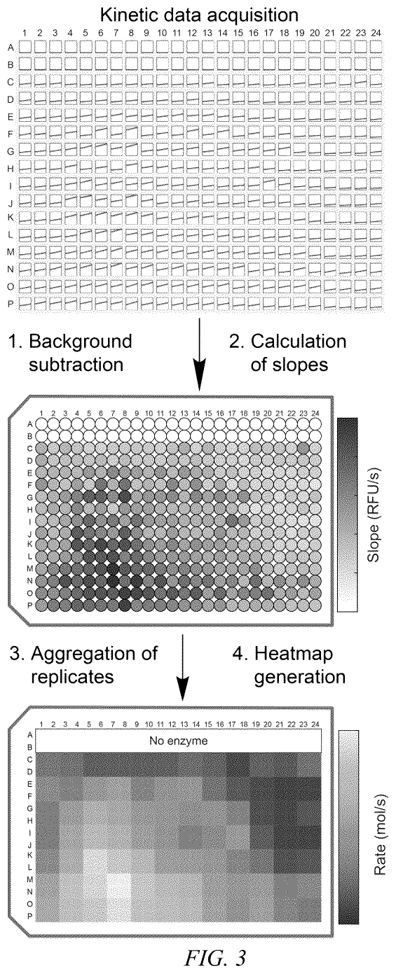

To assess the utility of CHESS to identify brush compositions that stabilize immobilized enzymes, we used microtiter well plates to systematically explore the compositional space of three-component copolymer brush supports. Specifically, we employed a spatially resolved polymerization pattern, varying SBMA:PEGMA horizontally in ˜9% increments from 0% to 100% PEGMA, where each column represented a particular SBMA/PEGMA ratio. Additionally, we varied the GMA concentration vertically (0.1-10%), so that each row contained a given GMA concentration. The plate was divided into squares of 2×2 wells to represent quadruplicates for each distinct copolymer composition, yielding 96 unique compositions, as detailed in Table 1. This plate arrangement allowed us to investigate optimal SBMA:PEGMA ratios (i.e., hydrophilic/hydrophobic balance), as well as the impact of GMA concentration (i.e., number of covalent attachment points) on immobilized enzyme performance. Following synthesis of brushes in the 384-well plate (i.e., with 96 distinct copolymer compositions) using green light, LipA was covalently immobilized to the brush layer in each well while omitting it from rows A and B (yielding 84 enzyme-containing polymer brush compositions). Following immobilization, the activity of LipA was assayed by tracking the release of the product for the fluorogenic substrate 4-methylumbelliferyl butyrate. Wells without enzyme were used to quantify background (spontaneous) substrate hydrolysis, which was subtracted from the hydrolysis of the enzyme-containing wells in the same column. By employing an automated code for high-throughput processing of fluorescence data and converting fluorescence intensity to product concentration using a calibration curve, the initial rate of LipA hydrolysis was estimated on a per well basis. Lastly, the rates for the same polymer compositions in each 2×2 well square were aggregated and the means of the aggregates were represented as a heat map. The overall workflow of the acquisition of kinetic activity data and data analysis is shown in FIG. 3. As shown in the initial rate heatmap in FIG. 3, regions on the plate corresponding to polymer brush compositions that supported higher enzyme activity than other compositions were clearly identified.

### Example 4—High-Throughput Screening of Thermally Stabilizing Brushes

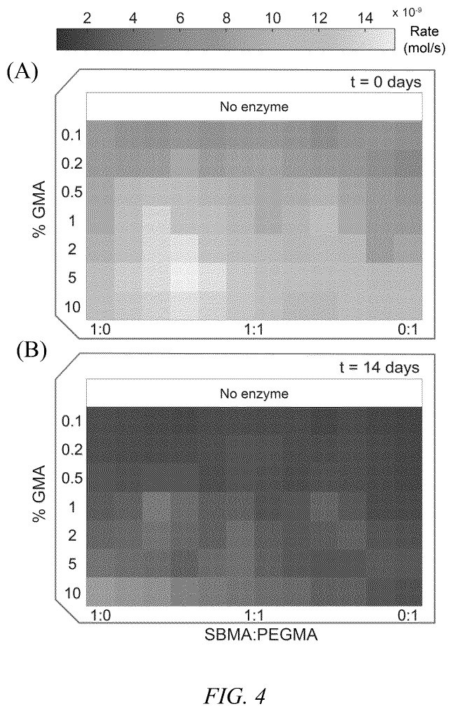

By enabling the high-throughput measurement of initial rate, we used CHESS to directly quantify the impact of polymer brush composition on LipA stability under thermally denaturing conditions. Specifically, we simultaneously probed the ability of 84 unique copolymer brush compositions to stabilize LipA at 50° C. by measuring activity as a function of incubation time (for 14 days total). Notably, activity was measured as a function of incubation time by sampling sequentially from the same wells to obtain multiple time points for three independent experimental replicates (data not shown). The results revealed that LipA immobilized on mixed compositions and intermediate GMA were most active at the initial time point, as indicated by the bright yellow region (light region in lower left of FIG. 4(A) when shown in grayscale) in the heatmap for intermediate compositions at t=Od (FIG. 4(A)). Specifically, brushes with intermediate PEGMA and GMA compositions (27% and 5%, respectively) exhibited the highest initial activity; however, immobilized LipA on these lost ˜85% of its initial activity over 14 days (FIG. 4(A)). In contrast, LipA immobilized on hydrophilic brushes with 0% PEGMA and 10% GMA exhibited exceptional activity retention over this time course despite showing lower initial activity. For comparison, the reduction in immobilized LipA activity was only half (˜43%) of that observed on the aforementioned brushes over the full-time course of the experiment. (FIG. 4(B)).

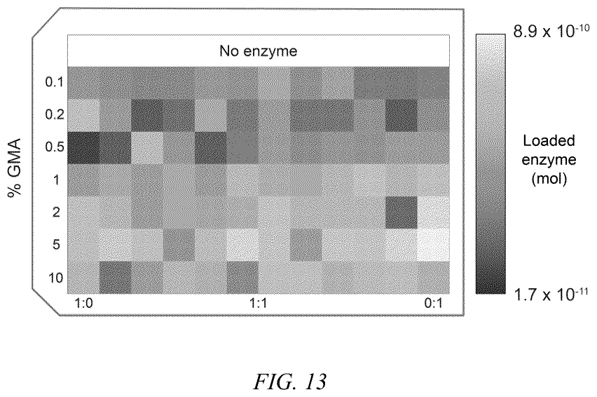

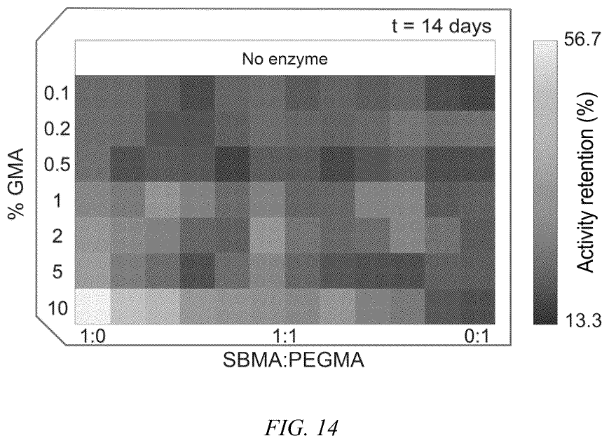

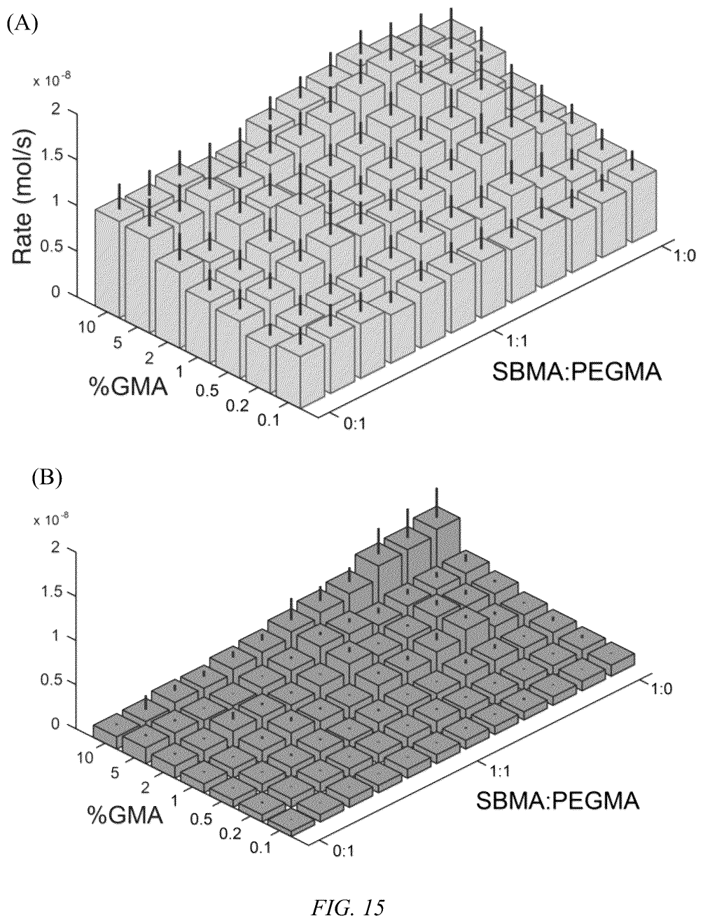

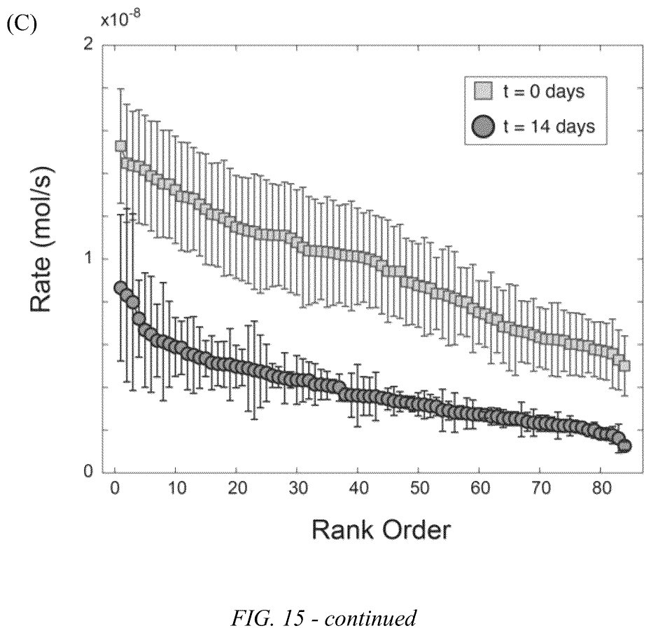

Importantly, differences in the absolute activity associated with LipA immobilized on different brush compositions may arise from not only the effects of the brush composition on enzyme activity, but also differences in enzyme loading in the wells. Because the loading of enzyme in the wells was low, the direct estimation of the amount of immobilized enzyme in each well was noisy (FIG. 13), making it impractical to normalize activity data by loading. However, any differences in loading could effectively be rendered negligible by analyzing relative changes in activity within each well, rather than analyzing absolute activity. The relative activity after 14 days for LipA immobilized on all 84 brush compositions (with respect to the initial activity) was plotted in FIG. 14. These high-throughput observations were consistent with our previous detailed studies, which found a trade-off between the initial LipA activity versus the long-term retention of LipA activity based on the number of enzyme-brush covalent attachments, where fewer attachments promoted higher initial activity and less stabilization, and vice-versa. Moreover, the observations aligned with previous results, which found that LipA was optimally stabilized when immobilized on 0% PEGMA brushes. Alternative representations of the data in FIG. 4 are shown in FIG. 18, which indicate the statistical uncertainty of the activity retention data based on well-to-well variation and independent replicates. In general, the statistical uncertainty was relatively consistent across the range of brush compositions, with standard deviations being approximately proportional to the mean values of LipA activity.

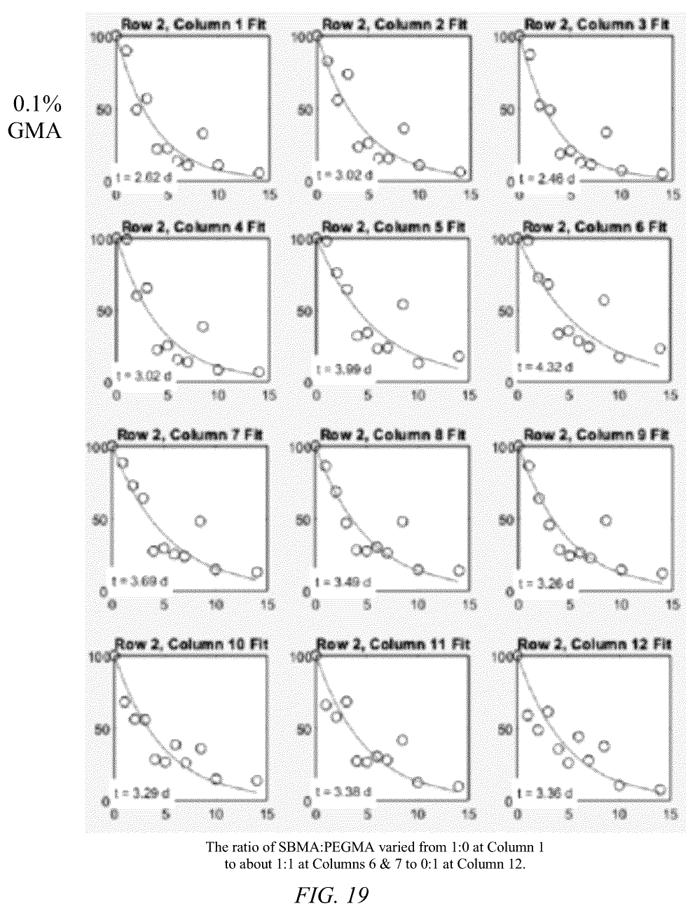

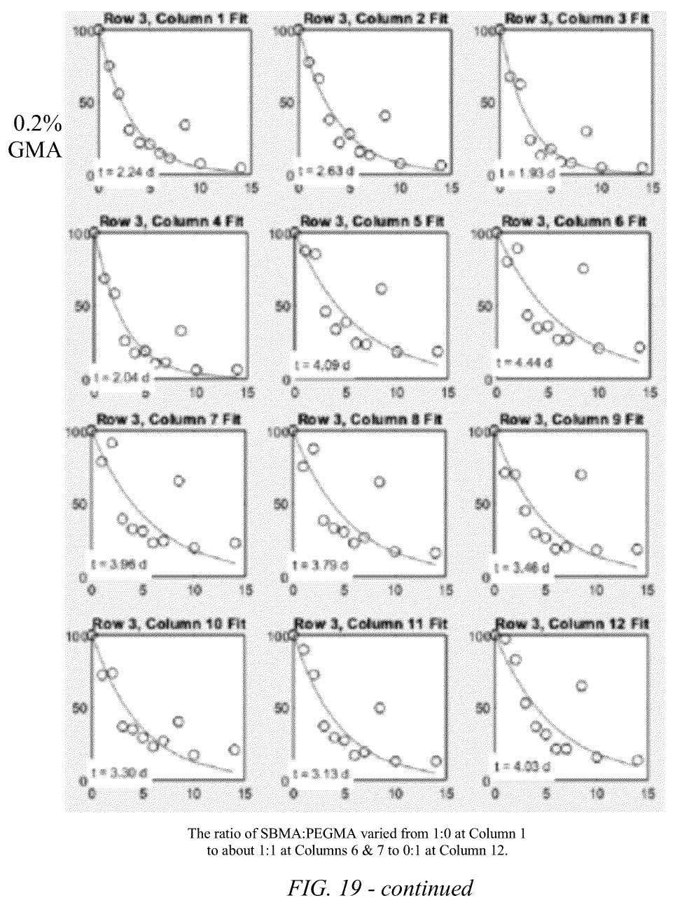

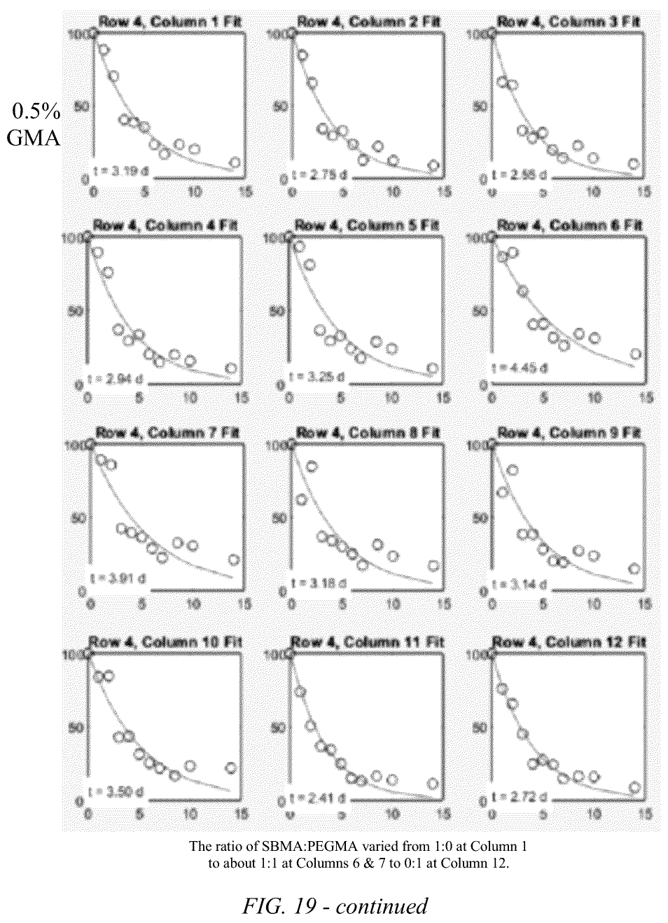

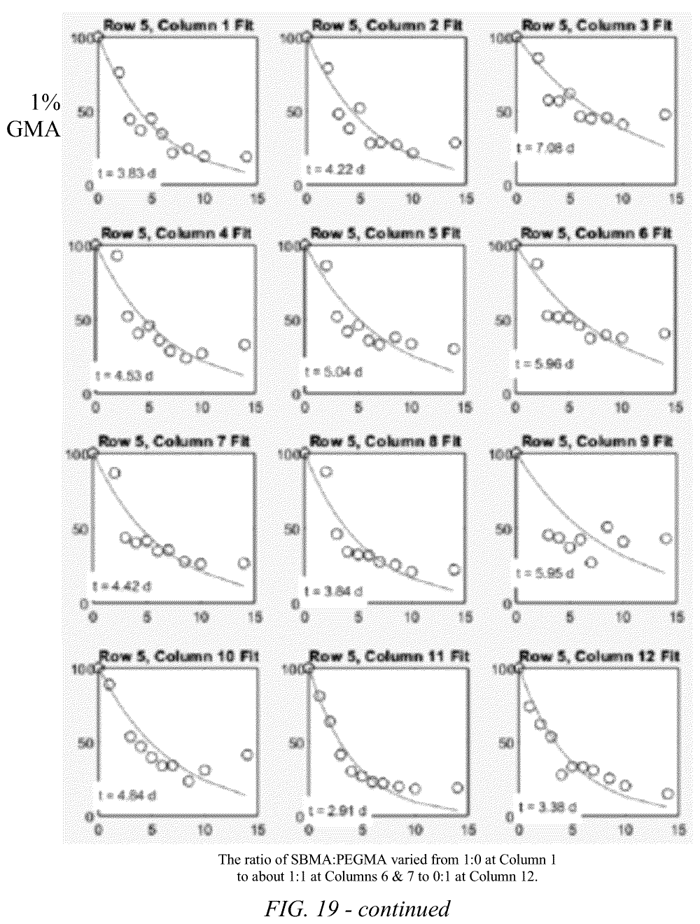

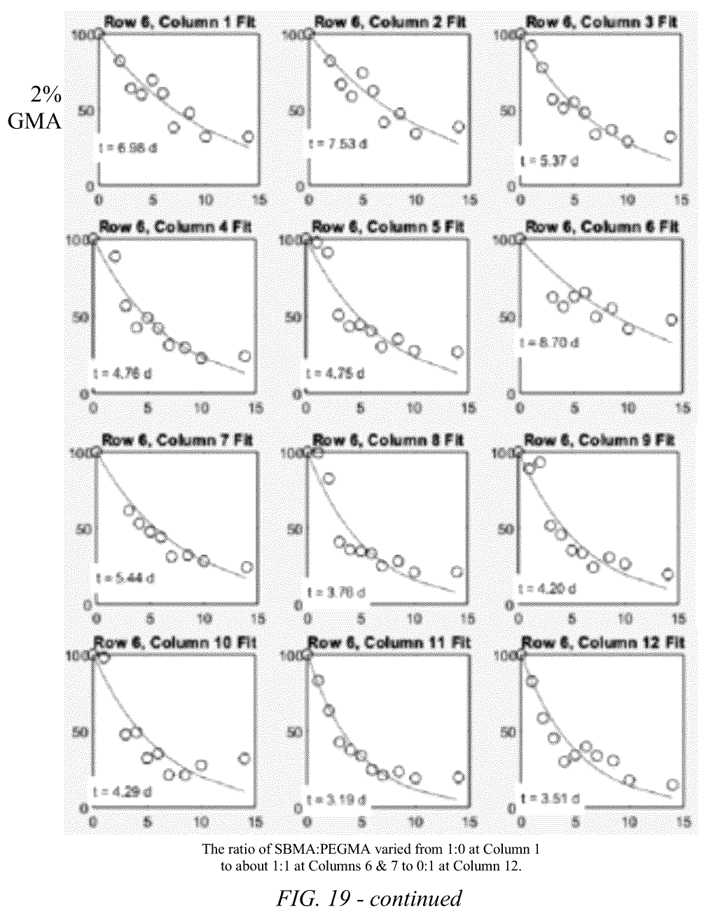

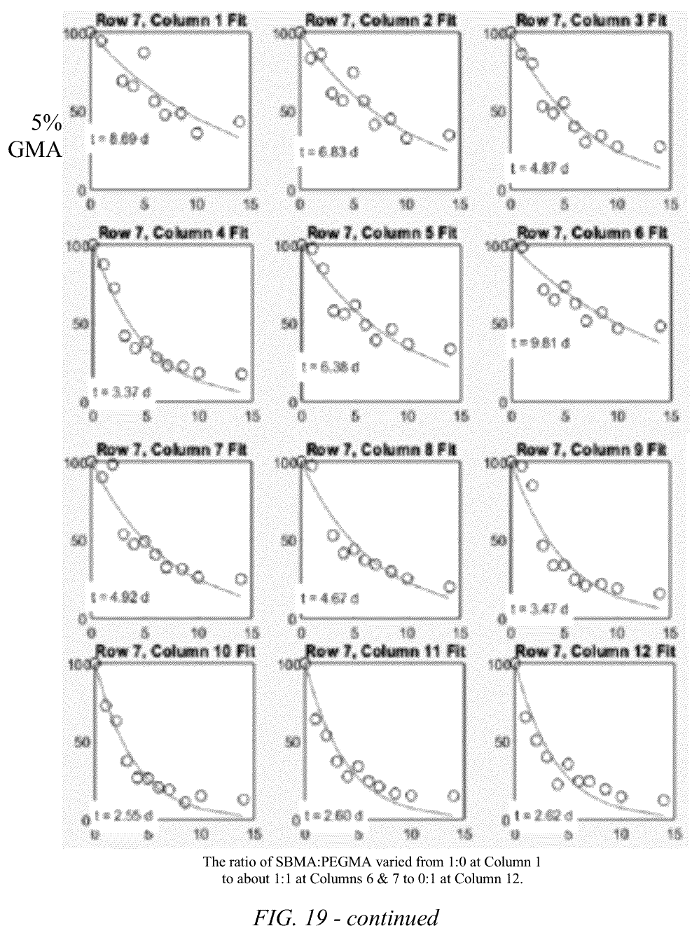

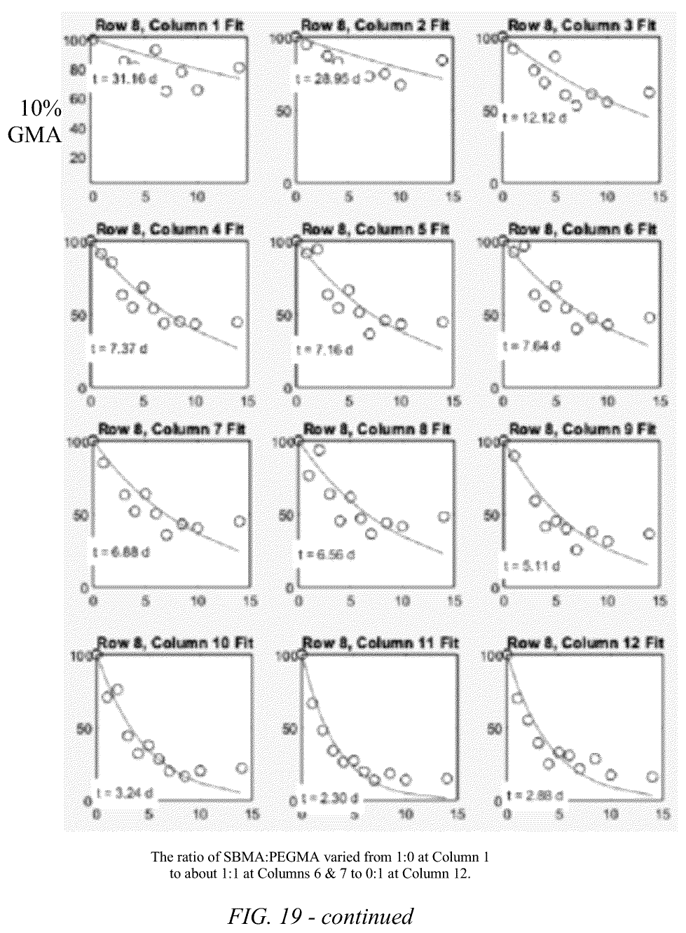

To further demonstrate the ability to measure the effect of brush composition on LipA deactivation kinetics, activity retention of immobilized LipA for each brush composition was analyzed as a function of time. For all wells, the deactivation profiles followed approximate first-order kinetics, which was consistent with prior findings showing that the deactivation kinetics of LipA in solution is first-order. After fitting each profile to a first-order exponential decay function (FIG. 19), the half-deactivation times (t_{1/2}) for LipA immobilized to each brush composition were determined and plotted as a heatmap (FIG. 5(A)). This analysis permitted a detailed visualization of LipA thermal stability over a wide range of deactivation rates. Consistent with our prior findings, the slowest deactivation (t_{1/2}=18.0±5.4 days) was observed for the composition previously noted as most stabilizing (10% GMA/0% PEGMA). Notably, CHESS enabled significant changes in deactivation kinetics due to subtle changes in brush composition to be identified and enabled a wide range of tin values to be measured. For example, LipA immobilized to a similar support with slightly more PEGMA (10% GMA/9% PEGMA) exhibited seemingly faster deactivation kinetics (t_{1/2}=13.9±6.1 days) compared to 0% PEGMA. The least stabilizing supports had t_{1/2} values of only ˜4 days, demonstrating the importance of tuning the support composition. As expected, a strong similarity was observed between the heatmaps for tin (FIG. 5(A)) and activity retention (FIG. 14), demonstrating that activity measurements at individual time points can be used to approximate detailed kinetic studies of enzyme deactivation.

To quantitatively validate CHESS as a reliable screening tool whose results accurately predict the performance of enzyme-immobilized biocatalyst particles, we selected five representative polymer brush compositions (labeled #1-5 in FIG. 5(A)) for a detailed comparison. We synthesized these copolymer brush support compositions using a similar polymerization approach on PP particles (previously coated with the 2BrBP initiator) and immobilized LipA using the same conjugation chemistry (i.e., via incorporation of GMA). Following immobilization on particles, we replicated the thermal deactivation experiments, fitted the data to first-order decay functions, and overlaid the results with the deactivation profiles determined from CHESS. As shown in FIG. 5(B), the thermal deactivation profiles for the biocatalyst particles showed good agreement with CHESS, with all data from the biocatalyst particles and their corresponding fits falling within the 95% prediction bands from the CHESS results. In addition to the good qualitative agreement between the well-plate and biocatalyst results, we found that the t_{1/2} values for particles and plate fits were similar (FIG. 5(B) annotation).

### Example 5—High-Throughput Screening of Brushes that Stabilize Against Chemical Denaturation

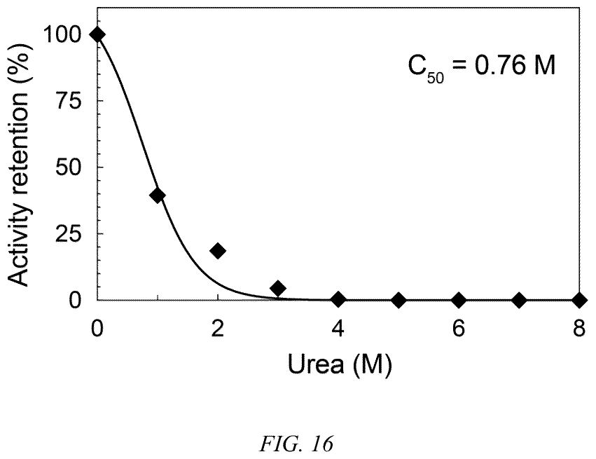

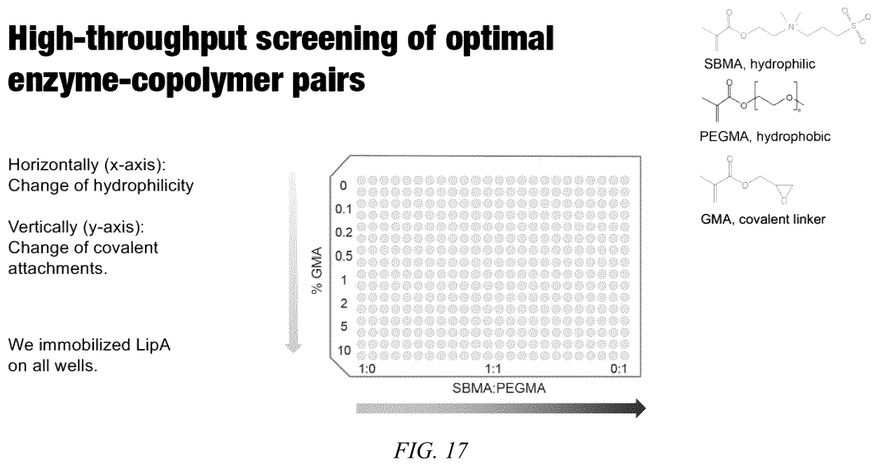

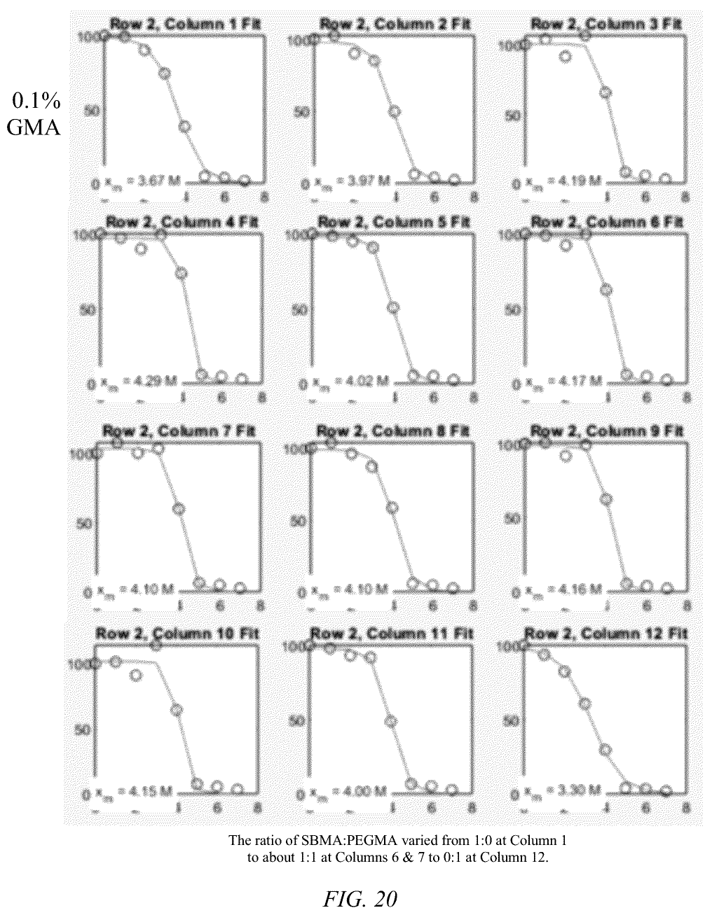

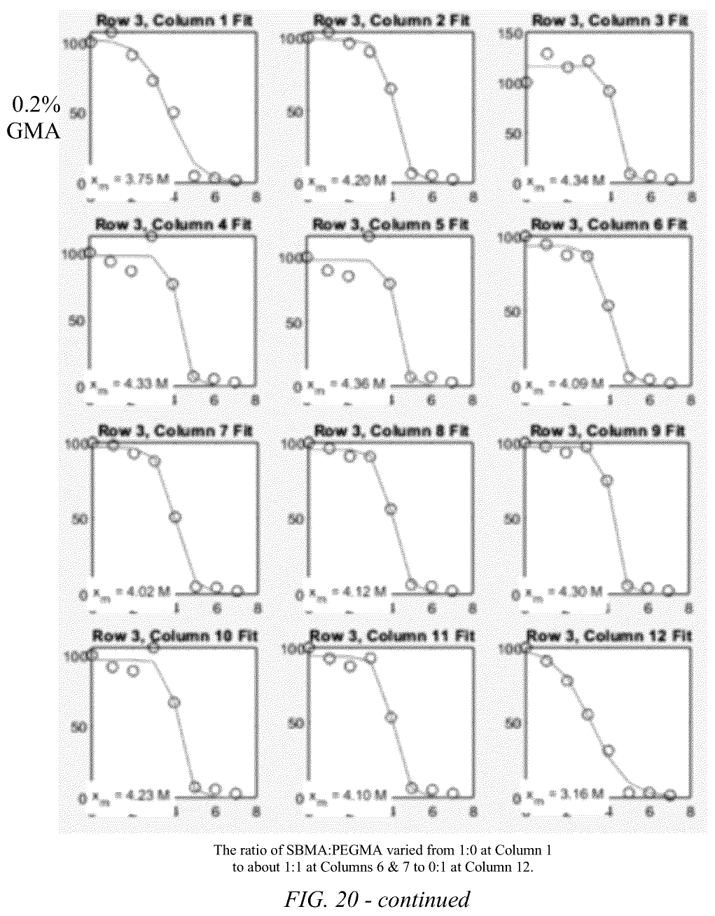

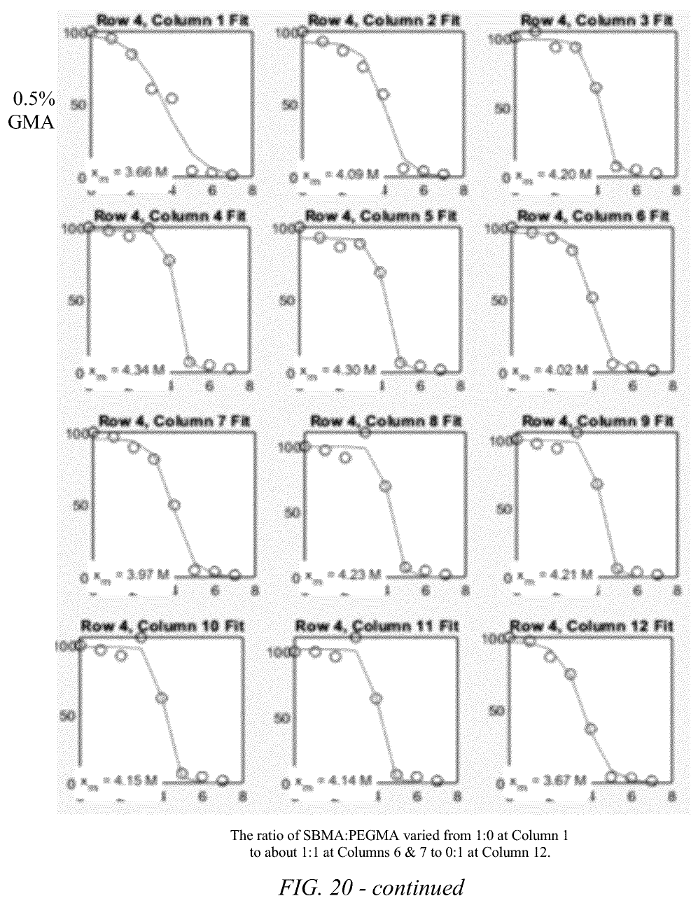

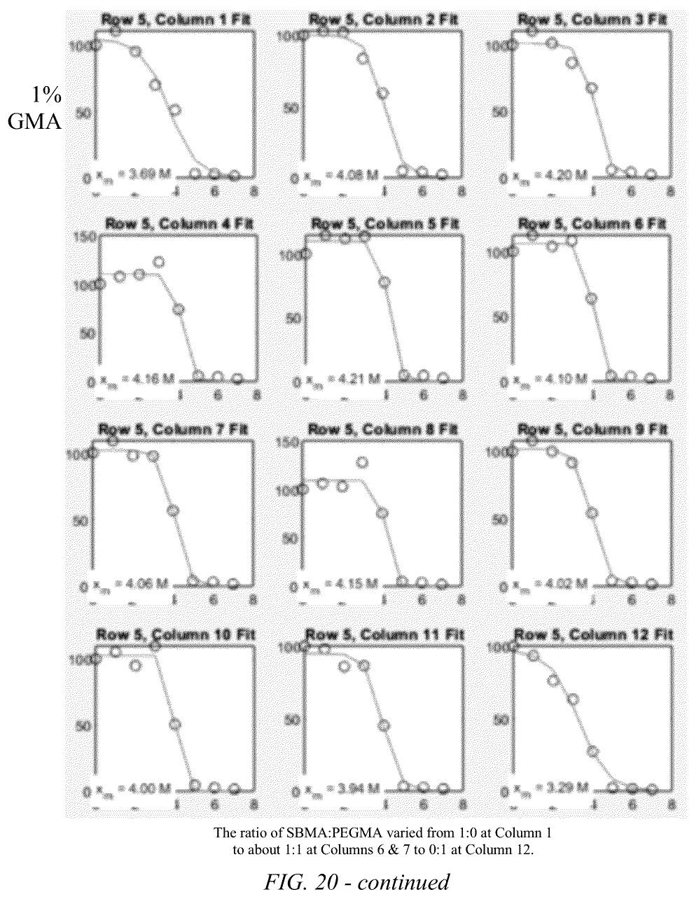

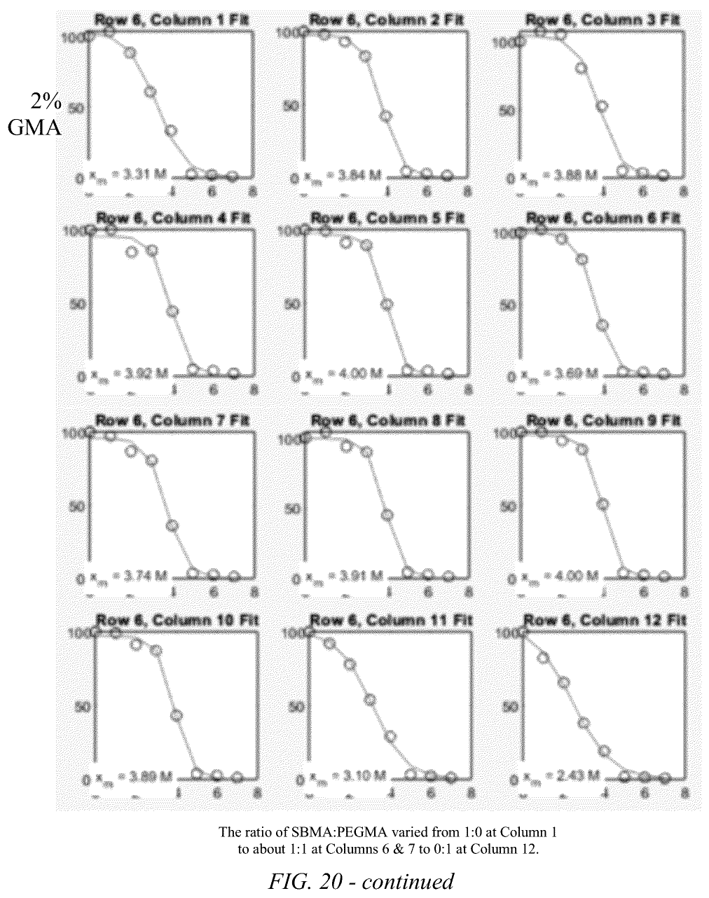

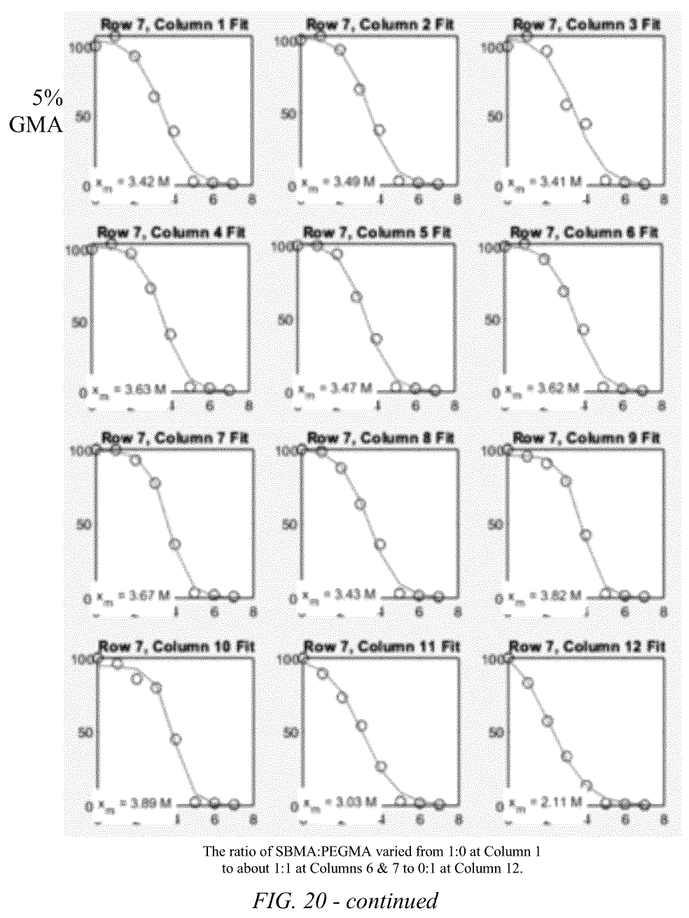

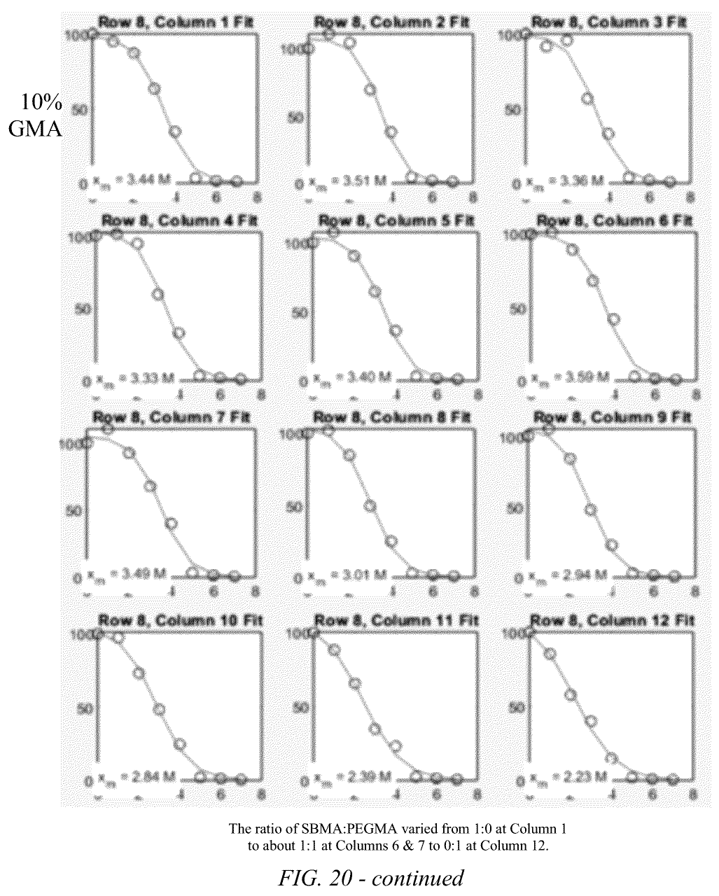

In addition to thermal denaturation, we also used CHESS to identify optimal random copolymer brush compositions that improve the resistance of LipA to chemical denaturation. For these experiments, LipA was immobilized to microtiter plates that had been modified with the same two-dimensional gradient of GMA and SBMA/PEGMA copolymers as described above, and LipA activity was measured after exposure to progressively higher concentrations of urea, ranging from 0 to 8 M in 1 M steps. For each urea increment, LipA was incubated for 3 h, its activity was assayed, and the buffer was exchanged to the subsequent higher urea concentration, using the same plate for successive assays. Analysis of activity data as a function of urea concentration indicated that the inactivation of LipA followed a sigmoidal curve, which is typical for urea-induced protein denaturation (FIG. 20). The data obtained from each well were fitted to a sigmoidal function, and the concentration for half-inactivation (C₅₀, which is the concentration of urea that resulted in a 50% loss of LipA activity relative to without urea) was determined for all 84 polymer brush compositions. Overall, the C₅₀ values for immobilized LipA spanned the range of 2.98-4.89 M urea, indicating that immobilization was consistently protective against chemical denaturation in all cases compared to soluble LipA, which had a C₅₀ of ˜0.76 M urea (FIG. 16).

By plotting the C₅₀ values as a heatmap (FIG. 6(A)), the effects of polymer brush composition on LipA stability towards chemical denaturation were readily visualized. Brush supports bearing low GMA content (with few enzyme-brush attachments) and mixed SBMA/PEGMA compositions (with intermediate hydrophilicity) were highly stabilizing against chemical denaturation, exhibiting values of C₅₀>4, as indicated by the bright yellow band (light gray in grayscale) across the top middle section of the heatmap (FIG. 6(A)). Polymer brushes with high GMA content were generally less stabilizing, with PEGMA conferring the lowest stability, as indicated by the dark blue region (light gray in grayscale) at lower right in the heatmap (FIG. 6a). To validate the reliability of CHESS to screen and predict the performance of enzyme-immobilized biocatalyst particles against chemical denaturation, we selected four compositions (#1-4) that were representative of different levels of stabilization across the composition gradient. Unexpectedly, the polymer composition regions yielding the highest C₅₀ was found to contain 0.2% GMA, where very few covalent attachments between the enzyme and the brush layer were expected. In terms of SBMA/PEGMA ratio, the polymer compositions that conferred the highest resistance against chemical denaturation (#1 and #2) were found in two regions at 27% (C₅₀=4.89±0.23 M) and 73% PEGMA (C₅₀=4.85±0.23 M), respectively. Interestingly, composition #3 (10% GMA/0% PEGMA), which was identified as the most stabilizing against thermal denaturation, yielded lower protection against chemical denaturation than compositions #1 and #2 with a C₅₀ value of 4.10 t 0.27 M. Accordingly, this may be explained by different mechanisms for protection against thermal and chemical denaturation by the polymer brush supports. Lastly, to include a wide range of support behaviors, support #4 was the worst performing support, with a C₅₀ value of 2.98±0.36 M.

As with the thermal denaturation case, we sought to determine if the findings from CHESS with chemical denaturation matched those from bulk assays with particles. This was determined by measuring the activity of LipA immobilized on polymer brush-modified PP particles with compositions #1-#4 over 0-8 M urea while incubating the particles for 3 h in buffer containing the target urea composition, similar to the measurements with CHESS. The results of the activity assays as a function of urea concentration for the particles were similarly fit with sigmoidal denaturation curves and are shown along with the data from the corresponding wells in FIG. 6(B). All sigmoidal fits for biocatalyst data were within the 95% prediction bands for the fit corresponding to the well-plate data, indicating strong agreement. Additionally, the paired C₅₀ values were very similar for both biocatalyst and well-plate data (FIG. 6(B) annotation), corroborating the validity of CHESS to capture chemical denaturation behavior that is transferrable to LipA immobilized on particles.

### Example 6—Statistical Analysis of the Comparison of CHESS with Particle Data

Although the above comparison of CHESS and particle data from thermal and chemical denaturation experiments suggests close agreement, we sought to more rigorously characterize the ability of CHESS to reproduce the behavior of immobilized biocatalyst particles. As such, the data from the thermal and chemical denaturation experiments were compared using several statistical metrics. For the thermal deactivation data, the Pearson correlation coefficient (rₜₕₑᵣₘₐₗ=0.925) suggested an excellent correlation between CHESS and particle measurements (FIG. 7(A)). To better evaluate the absolute agreement between the well-plate and biocatalyst data, we calculated the Intraclass Correlation Coefficient (ICC) which explicitly evaluates agreement between two methods. The ICC was 0.827, indicating good agreement, but lower than suggested by rₜₕₑᵣₘₐₗ. To understand this apparent discrepancy, we performed Bland-Altman analysis (FIG. 7(B)), which revealed that the CHESS method tended to overestimate activity retention data compared to biocatalyst particles for thermal denaturation experiments, with a bias of 5.2% in enzymatic activity retention. For chemical denaturation experiments, Pearson analysis indicated a remarkable correlation (r_{chemical}=0.973) (FIG. 7(C)) and ICC also suggested excellent reliability with 0.937; however, an offset in their values also suggested some bias for chemical deactivation experiments. Using Bland-Altman analysis, we determined a minor systematic underestimation of activity retention by CHESS, with a mean bias of −4.6% (FIG. 7d). Overall, these statistical methods corroborated that CHESS is a highly reliable methodology that accurately replicates the behavior of immobilized enzymes on biocatalyst particles under both thermal and chemical denaturing conditions.

### Example 7—Screening of Optimally Stabilizing Brush Compositions for AlkP

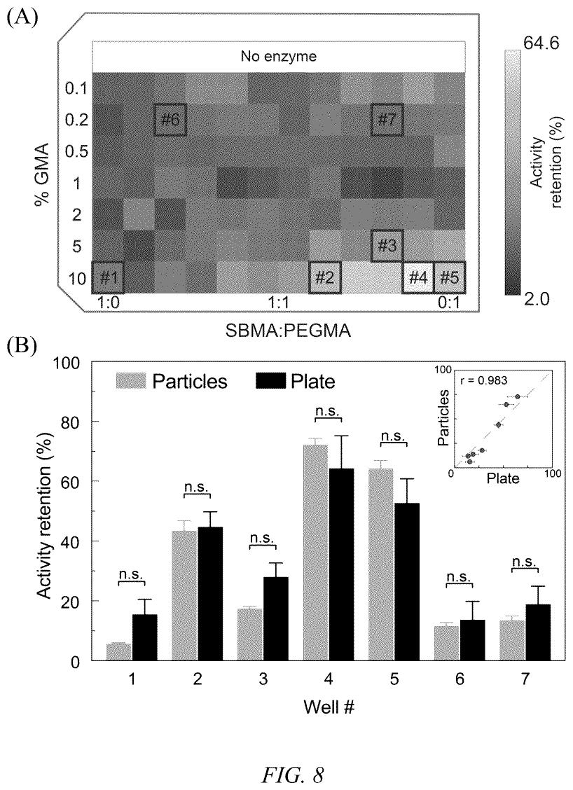

To demonstrate the generality of this high-throughput approach, we used CHESS to identify stabilizing brush compositions for AlkP. Similar to LipA, AlkP is widely used in the food and biotechnology industries and thus is of high industrial relevance, yet has not been studied previously on mixed PEGMA/SBMA brush supports. Therefore, a priori knowledge of the optimal ratio of PEGMA-to-SBMA was not known prior to screening of immobilized AlkP with CHESS. Following immobilization of AlkP to the polymer-modified well-plate as described above for LipA, its initial activity in each well was measured using 4-methylumbelliferyl phosphate as a substrate. Subsequently, the plate containing AlkP with buffer in each well was incubated at 50° C. for 88 h to simulate thermal stress under operational conditions. After incubation, the wells were replenished with fresh buffer and residual AlkP activity was assayed, yielding the activity retention heatmap shown in FIG. 8(A). Interestingly, the heatmap revealed a region in the SBMA/PEGMA/GMA compositional space with exceptional thermal stabilization properties (in grayscale it appears as a lightgray to while region at lower right in FIG. 8(A)), with immobilized AlkP retaining as much as ˜65% of its initial activity, whereas AlkP was almost completely inactivated on other regions. The brush compositions that promoted high AlkP thermal stabilization corresponded to 91% PEGMA and 10% GMA, indicating that AlkP was stabilized by the formation of many covalent tethers between the enzyme and the brush layer.

From the CHESS data with immobilized AlkP, we selected seven representative polymer compositions (#1-7), synthesized biocatalyst particles with those same compositions, and immobilized AlkP on each (black square outlines on FIG. 8(A)). To test a wide range of support compositions, some of the selected supports had compositions that were located near the high thermal stabilization region of compositional space (#2, #3, #4, and #5), whereas others were scattered throughout non-stabilizing regions (#1, #6, and #7). To enable comparison with data from CHESS, the activity of immobilized AlkP on brush-modified PP particles was measured under identical conditions (i.e., incubation at 50° C. for 88 h). The comparison between activity retention for AlkP biocatalyst particles and immobilized to a well-plate is shown in FIG. 8(B). As for LipA, we observed excellent agreement between CHESS and particle data, and individual t-tests for each composition pair revealed no statistically significant differences between the measurements at identical brush compositions. Additionally, analysis of the correlation of the measurements found a Pearson coefficient of r=0.983. While enabling the identification of optimal brush supports to preserve AlkP activity at elevated temperatures, these findings further demonstrated the ability of CHESS to reliably match data from conventional screening approaches and enable the discovery of novel immobilized biocatalysts with remarkably stabilizing properties.

In addition to validation with biocatalyst particles, we sought to computationally validate the findings by analyzing the surface properties of AlkP using Hi-patch, which employs an algorithm to quantify the extent and hydrophobic intensity of protein surfaces. We found that AlkP's surface is remarkably hydrophobic with a free energy of solvation of −11.3 kJ/mol·nm². Using the experimentally determined correlation between surface hydrophobicity and preferred SBMA/PEGMA copolymer brush composition support from our prior work, the estimated free energy of solvation for AlkP suggested it would be preferentially stabilized on supports that were more hydrophobic (i.e., with higher a PEGMA fraction), which was consistent with the observations from CHESS. For comparison, the free energy of solvation of LipA is −15.5 kJ/mol·nm², which suggests it has a more hydrophilic surface, consistent with its stabilization on SBMA-rich hydrophilic supports that was observed previously as well as in this work.

In summary, we described a novel platform (CHESS) that enables rapid screening of copolymer brush supports bearing multiple different monomers for enzyme immobilization and enables the rapid and facile discovery of biocompatible materials. Using the exogenous initiator molecule 2BrBP to functionalize the walls of 384-well PP microplates, we were able to successfully grow random copolymer brushes inside individual wells of the microplate while simultaneously varying compositions in two-dimensions. By incorporating monomers with protein-reactive handles, we demonstrated that enzymes could be immobilized to the brush layer within the wells and screened for immobilized enzyme activity and stability in situ. From thermal and chemical denaturation experiments, extracted t_{1/2} and C₅₀ values, respectively. These values showed remarkably close agreement to those obtained from conventional bulk assays with enzyme immobilized on particles, thereby highlighting the reliability and reproducibility of CHESS to replicate the results from traditional screening approaches. We further used CHESS to also discover a support composition that was exceptionally thermally stabilizing for AlkP, an enzyme that had not previously been immobilized to polymer brush supports. These findings illustrate the potential of the well-plate-based CHESS method for the discovery of new immobilization supports for applications in biotechnology, notably immobilized biocatalysis. Considering the increasing complexity of biochemical reactions and industrial demands, the application of CHESS can rapidly enable the adaptation of unstable enzymes that are sensitive to harsh conditions by screening complex multi-component immobilization supports bearing moieties that enable complex covalent and non-covalent interactions (e.g., charged, hydrophobic, H-bonding) between the enzyme and the brush layer.

### Glossary of Claim Terms

As used throughout the entire application, the terms “a” and “an” are used in the sense that they mean “at least one”, “at least a first”, “one or more” or “a plurality” of the referenced components or steps, unless the context clearly dictates otherwise. For example, the term “a cell” includes a plurality of cells, including mixtures thereof.

The term “and/or” wherever used herein includes the meaning of “and”, “or” and “all or any other combination of the elements connected by said term”.

The term “about” or “approximately” as used herein means within 20%, preferably within 10%, and more preferably within 5% of a given value or range.

Other than in the operating examples, or unless otherwise expressly specified, all of the numerical ranges, amounts, values and percentages such as those for amounts of materials, times and temperatures of reaction, ratios of amounts, values for molecular weight (whether number average molecular weight (“Mₙ”) or weight average molecular weight (“M_{w}”), and others in the following portion of the specification may be read as if prefaced by the word “about” even though the term “about” may not expressly appear with the value, amount or range. Accordingly, unless indicated to the contrary, the numerical parameters set forth in the following specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical value, however, inherently contain certain errors necessarily resulting from the standard deviation found in their respective testing measurements. Furthermore, when numerical ranges of varying scope are set forth herein, it is contemplated that any combination of these values inclusive of the recited values may be used.

As used herein, the term “composition” is intended to encompass a product comprising the specified ingredients in the specified amounts, as well as any product which results, directly or indirectly, from combination of the specified ingredients in the specified amounts. A polymer is a chemical compound or mixture of compounds formed by polymerization and consisting essentially of repeating structural units (e.g., a monomer). A monomer is a molecule that can be bonded to other identical molecules to form a polymer. A homopolymer is a polymer that is made up of only one type of monomer unit. A copolymer is a polymer formed when two (or more) different types of monomers are linked in the same polymer chain (as opposed to a homopolymer where only one monomer is used). A statistical copolymer is a polymer in which two or more monomers are arranged in a sequence that follows some statistical rule. If the mole fraction of a monomer be equal to the probability of finding a residue of that monomer at any point in the chain, the polymer is a random polymer. These polymers are generally synthesized via the free radical polymerization method.

As used herein, the term “base material” refers to a substrate providing one or more surfaces, where the surface is capable of forming polymer brushes, or to which polymer brushes can be grafted or otherwise affixed.

As used herein, the term “brush” or “polymer brush” refers to a polymeric side chain that is formed from a polymerization substrate having a radical-polymerizable terminal group, wherein the polymerizable substrate is the base material, or can be engrafted to or otherwise affixed to the base material, thereby substantially taking the form of the base material.

As used herein the term “reactive monomer” refers to a compound that is capable of participating in a radical induced grafting reaction. The reactive monomer can be any material capable of forming polymers as described above and herein, for example but not limited to glycidyl methacrylate (GMA), or ethylene. The base material and reactive monomer may be of the same compound, for example, a polyethylene base material may utilize ethelyene monomers or polymers in the grafting reaction.

As used herein, the term “comprising” is intended to mean that the products, compositions and methods include the referenced components or steps, but not excluding others. “Consisting essentially of” when used to define products, compositions and methods, shall mean excluding other components or steps of any essential significance. Thus, a composition consisting essentially of the recited components would not exclude trace contaminants and pharmaceutically acceptable carriers. “Consisting of” shall mean excluding more than trace elements of other components or steps.

The advantages set forth above, and those made apparent from the foregoing description, are efficiently attained. Since certain changes may be made in the above construction without departing from the scope of the invention, it is intended that all matters contained in the foregoing description or shown in the accompanying drawings shall be interpreted as illustrative and not in a limiting sense.

All references cited in the present application are incorporated in their entirety herein by reference to the extent not inconsistent herewith.

It will be seen that the advantages set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall therebetween. Now that the invention has been described,

TABLE 1

| | 1 | 2 | 3 | 4 | 5 | 6 | 7 | 8 | 9 | 10 | 11 | 12 |
|---|---|---|---|---|---|---|---|---|---|---|---|---|
| A | 100.0 | % SBMA | 90.9 | % SBMA | 81.8 | % SBMA | 72.7 | % SBMA | 63.6 | % SBMA | 54.5 | % SBMA |
| B | 0.0 | % PEGMA | 9.1 | % PEGMA | 18.2 | % PEGMA | 27.3 | % PEGMA | 36.4 | % PEGMA | 45.5 | % PEGMA |
| | 0 | % GMA | 0 | % GMA | 0 | % GMA | 0 | % GMA | 0 | % GMA | 0 | % GMA |
| C | 100.0 | % SBMA | 90.9 | % SBMA | 81.8 | % SBMA | 72.7 | % SBMA | 63.6 | % SBMA | 54.5 | % SBMA |
| D | 0.0 | % PEGMA | 9.1 | % PEGMA | 18.2 | % PEGMA | 27.3 | % PEGMA | 36.4 | % PEGMA | 45.5 | % PEGMA |
| | 0.1 | % GMA | 0.1 | % GMA | 0.1 | % GMA | 0.1 | % GMA | 0.1 | % GMA | 0.1 | % GMA |
| E | 100.0 | % SBMA | 90.9 | % SBMA | 81.8 | % SBMA | 72.7 | % SBMA | 63.6 | % SBMA | 54.5 | % SBMA |
| F | 0.0 | % PEGMA | 9.1 | % PEGMA | 18.2 | % PEGMA | 27.3 | % PEGMA | 36.4 | % PEGMA | 45.5 | % PEGMA |
| | 0.2 | % GMA | 0.2 | % GMA | 0.2 | % GMA | 0.2 | % GMA | 0.2 | % GMA | 0.2 | % GMA |
| G | 100.0 | % SBMA | 90.9 | % SBMA | 81.8 | % SBMA | 72.7 | % SBMA | 63.6 | % SBMA | 54.5 | % SBMA |
| H | 0.0 | % PEGMA | 9.1 | % PEGMA | 18.2 | % PEGMA | 27.3 | % PEGMA | 36.4 | % PEGMA | 45.5 | % PEGMA |
| | 0.5 | % GMA | 0.5 | % GMA | 0.5 | % GMA | 0.5 | % GMA | 0.5 | % GMA | 0.5 | % GMA |
| I | 100.0 | % SBMA | 90.9 | % SBMA | 81.8 | % SBMA | 72.7 | % SBMA | 63.6 | % SBMA | 54.5 | % SBMA |
| J | 0.0 | % PEGMA | 9.1 | % PEGMA | 18.2 | % PEGMA | 27.3 | % PEGMA | 36.4 | % PEGMA | 45.5 | % PEGMA |
| | 1 | % GMA | 1 | % GMA | 1 | % GMA | 1 | % GMA | 1 | % GMA | 1 | % GMA |
| K | 100.0 | % SBMA | 90.9 | % SBMA | 81.8 | % SBMA | 72.7 | % SBMA | 63.6 | % SBMA | 54.5 | % SBMA |
| L | 0.0 | % PEGMA | 9.1 | % PEGMA | 18.2 | % PEGMA | 27.3 | % PEGMA | 36.4 | % PEGMA | 45.5 | % PEGMA |
| | 2 | % GMA | 2 | % GMA | 2 | % GMA | 2 | % GMA | 2 | % GMA | 2 | % GMA |
| M | 100.0 | % SBMA | 90.9 | % SBMA | 81.8 | % SBMA | 72.7 | % SBMA | 63.6 | % SBMA | 54.5 | % SBMA |
| N | 0.0 | % PEGMA | 9.1 | % PEGMA | 18.2 | % PEGMA | 27.3 | % PEGMA | 36.4 | % PEGMA | 45.5 | % PEGMA |
| | 5 | % GMA | 5 | % GMA | 5 | % GMA | 5 | % GMA | 5 | % GMA | 5 | % GMA |
| P | 100.0 | % SBMA | 90.9 | % SBMA | 81.8 | % SBMA | 72.7 | % SBMA | 63.6 | % SBMA | 54.5 | % SBMA |
| O | 0.0 | % PEGMA | 9.1 | % PEGMA | 18.2 | % PEGMA | 27.3 | % PEGMA | 36.4 | % PEGMA | 45.5 | % PEGMA |
| | 10 | % GMA | 10 | % GMA | 10 | % GMA | 10 | % GMA | 10 | % GMA | 10 | % GMA |
| | 13 | 14 | 15 | 16 | 17 | 18 | 19 | 20 | 21 | 22 | 23 | 24 |
| A | 45.5 | % SBMA | 36.4 | % SBMA | 27.3 | % SBMA | 18.2 | % SBMA | 9.1 | % SBMA | 0.0 | % SBMA |
| B | 54.5 | % PEGMA | 63.6 | % PEGMA | 72.7 | % PEGMA | 81.8 | % PEGMA | 90.9 | % PEGMA | 100.0 | % PEGMA |
| | 0 | % GMA | 0 | % GMA | 0 | % GMA | 0 | % GMA | 0 | % GMA | 0 | % GMA |
| C | 45.5 | % SBMA | 36.4 | % SBMA | 27.3 | % SBMA | 18.2 | % SBMA | 9.1 | % SBMA | 0.0 | % SBMA |
| D | 54.5 | % PEGMA | 63.6 | % PEGMA | 72.7 | % PEGMA | 81.8 | % PEGMA | 90.9 | % PEGMA | 100.0 | % PEGMA |
| | 0.1 | % GMA | 0.1 | % GMA | 0.1 | % GMA | 0.1 | % GMA | 0.1 | % GMA | 0.1 | % GMA |
| E | 45.5 | % SBMA | 36.4 | % SBMA | 27.3 | % SBMA | 18.2 | % SBMA | 9.1 | % SBMA | 0.0 | % SBMA |
| F | 54.5 | % PEGMA | 63.6 | % PEGMA | 72.7 | % PEGMA | 81.8 | % PEGMA | 90.9 | % PEGMA | 100.0 | % PEGMA |
| | 0.2 | % GMA | 0.2 | % GMA | 0.2 | % GMA | 0.2 | % GMA | 0.2 | % GMA | 0.2 | % GMA |
| G | 45.5 | % SBMA | 36.4 | % SBMA | 27.3 | % SBMA | 18.2 | % SBMA | 9.1 | % SBMA | 0.0 | % SBMA |
| H | 54.5 | % PEGMA | 63.6 | % PEGMA | 72.7 | % PEGMA | 81.8 | % PEGMA | 90.9 | % PEGMA | 100.0 | % PEGMA |
| | 0.5 | % GMA | 0.5 | % GMA | 0.5 | % GMA | 0.5 | % GMA | 0.5 | % GMA | 0.5 | % GMA |
| I | 45.5 | % SBMA | 36.4 | % SBMA | 27.3 | % SBMA | 18.2 | % SBMA | 9.1 | % SBMA | 0.0 | % SBMA |
| J | 54.5 | % PEGMA | 63.6 | % PEGMA | 72.7 | % PEGMA | 81.8 | % PEGMA | 90.9 | % PEGMA | 100.0 | % PEGMA |
| | 1 | % GMA | 1 | % GMA | 1 | % GMA | 1 | % GMA | 1 | % GMA | 1 | % GMA |
| K | 45.5 | % SBMA | 36.4 | % SBMA | 27.3 | % SBMA | 18.2 | % SBMA | 9.1 | % SBMA | 0.0 | % SBMA |
| L | 54.5 | % PEGMA | 63.6 | % PEGMA | 72.7 | % PEGMA | 81.8 | % PEGMA | 90.9 | % PEGMA | 100.0 | % PEGMA |
| | 2 | % GMA | 2 | % GMA | 2 | % GMA | 2 | % GMA | 2 | % GMA | 2 | % GMA |
| M | 45.5 | % SBMA | 36.4 | % SBMA | 27.3 | % SBMA | 18.2 | % SBMA | 9.1 | % SBMA | 0.0 | % SBMA |
| N | 54.5 | % PEGMA | 63.6 | % PEGMA | 72.7 | % PEGMA | 81.8 | % PEGMA | 90.9 | % PEGMA | 100.0 | % PEGMA |
| | 5 | % GMA | 5 | % GMA | 5 | % GMA | 5 | % GMA | 5 | % GMA | 5 | % GMA |
| P | 45.5 | % SBMA | 36.4 | % SBMA | 27.3 | % SBMA | 18.2 | % SBMA | 9.1 | % SBMA | 0.0 | % SBMA |
| O | 54.5 | % PEGMA | 63.6 | % PEGMA | 72.7 | % PEGMA | 81.8 | % PEGMA | 90.9 | % PEGMA | 100.0 | % PEGMA |
| | 10 | % GMA | 10 | % GMA | 10 | % GMA | 10 | % GMA | 10 | % GMA | 10 | % GMA |

1. A method of screening copolymer compositions as supports for enzymes comprising the steps of:
providing a bis[bromo]benzophenone-functionalized (2BrBP-functionalized) multi-well plate wherein a plurality of the wells of the multi-well plate have a defined and unique copolymer brush affixed to the bis[bromo]benzophenone (2BrBP) in a well and wherein the copolymer brush is created by systematically varying the relative concentration of the constituent monomers or other constituents used to create the copolymer brush, whereby the performance of the protein of interest can then be assayed in the unique polymer brush environment of the well;
adding an enzyme to the copolymer brushes of the wells;
adding an enzyme substrate to the wells;
exposing the plate to a defined set of conditions or chemicals; and
measuring the conversion of the enzyme substrate in each of the plurality of wells responsive to the exposure to the defined set of conditions or chemicals.

2. The method according to claim 1 wherein the copolymer brush is formed from an aromatic monomer in combination with a non-aromatic monomer, wherein the co-polymer brush is adapted to stabilize an enzyme to be linked thereto.

3. The method according to claim 2 wherein the aromatic monomer is selected from the group consisting of ethylene glycol phenyl ether methacrylate (EGPMA), phenyl methacrylate, benzyl methacrylate, diethylene glycol phenyl ether methacrylate, triethylene glycol phenyl ether methacrylate, tetraethylene glycol phenyl ether methacrylate, pentaethylene glycol phenyl ether methacrylate, poly(ethylene glycol) phenyl ether methacrylate, 1-Naphthyl methacrylate, 2-Naphthyl methacrylate, 1-Pyrenemethyl methacrylate, 9-Anthracenemethyl methacrylate, styrene, ethylene glycol phenyl ether acrylate, phenyl acrylate, benzyl acrylate, diethylene glycol phenyl ether acrylate, triethylene glycol phenyl ether acrylate, tetraethylene glycol phenyl ether acrylate, pentaethylene glycol phenyl ether acrylate, poly(ethylene glycol) phenyl ether acrylate, 1-Naphthyl acrylate, 2-Naphthyl acrylate, 1-Pyrenemethyl acrylate, 9-Anthracenemethyl acrylate, ethylene glycol phenyl ether acrylamide, phenyl acrylamide, benzyl acrylamide, diethylene glycol phenyl ether acrylamide, triethylene glycol phenyl ether acrylamide, tetraethylene glycol phenyl ether acrylamide, pentaethylene glycol phenyl ether acrylamide, poly(ethylene glycol) phenyl ether acrylamide, 1-Naphthyl acrylamide, 2-Naphthyl acrylamide, 1-Pyrenemethyl acrylamide and 9-Anthracenemethyl acrylamide, ethylene glycol phenyl ether methacrylamide, phenyl methacrylamide, benzyl methacrylamide, diethylene glycol phenyl ether methacrylamide, triethylene glycol phenyl ether methacrylamide, tetraethylene glycol phenyl ether methacrylamide, pentaethylene glycol phenyl ether methacrylamide, poly(ethylene glycol) phenyl ether methacrylamide, 1-Naphthyl methacrylamide, 2-Naphthyl methacrylamide, 1-Pyrenemethyl methacrylamide and 9-Anthracenemethyl methacrylamide.

4. The method according to claim 2 wherein the non-aromatic monomer is selected from the group consisting of sulfobetaine methacrylate (SBMA), Phosphorylcholine methacrylate (PCMA), Glycosyloxyethyl methacrylate (GEMA), poly(ethylene glycol) methacrylate (PEGMA).

5. The method according to claim 2 wherein the non-aromatic monomer is selected from the group consisting of [3-(methacryloylamino)propyl]trimethylammonium chloride, [2-(methacryloyloxy)ethyl)]trimethylammonium chloride, 3-sulfopropyl methacrylate, 3-sulfopropyl acrylate, 3-sulfopropyl acrylamide, 3-sulfopropyl methacrylamide, [2-(methacryloyloxy)ethyl]dimethyl-(3-sulfopropyl)ammonium hydroxide, methacrylic acid, acrylic acid, acrylamide, methacrylamide, poly(ethylene glycol) methacrylate, poly(ethylene glycol) acrylate, poly(ethylene glycol) acrylamide, poly(ethylene glycol) methacrylamide, 2-(diethylamino)ethyl methacrylate, 2-(diethylamino)ethyl acrylate, 2-(diethylamino)ethyl acrylamide, 2-(diethylamino)ethyl methacrylamide, n-isopropylacrylamide, 2-N-morpholinoethyl methacrylate, 2-N-morpholinoethyl acrylate, 2-N-morpholinoethyl acrylamide, and 2-N-morpholinoethyl methacrylamide.

6. The method according to claim 2 wherein the non-aromatic monomer is a zwitterionic monomer.

7. The method according to claim 6 wherein the zwitterionic monomer is selected from the group consisting of sulfobetaine acrylate, sulfobetaine acrylamide, sulfobetaine methacrylamide, phosphorylcholine acrylate, phosphorylcholine acrylamide, phosphorylcholine methacrylamide, acryloyl serine, acryloyl ornithine, acryloyl lysine, and acryloyl glutamate.

8. The method according to claim 1 wherein the conversion of substrate is measured with colorimetric or fluorometric assays.

9. The method according to claim 1 wherein the enzyme is selected from the groups lipase, carbonic anhydrase, cytochrome P450, benzaldehyde lyase, alkaline phosphatase, trypsin, chymotrypsin, thrombin, subtilisin, horseradish peroxidase, acetylcholinesterase, glucose isomerase, penicillin g acylase, epimerase, phytase, protein A, transaminase, nitroreductase, unspecific peroxygenase, and imine reductase.

10. The method according to claim 1 wherein the enzyme is selected from the group consisting of Candida rugosa lipase (CRL), Candida antarctica lipase B (CALB), Rhizomucor miehei lipase (RML), Bacillus subtilis lipase A (LipA), Pseudomonas stutzeri triacylglycerol lipase (lipase TL), and lipase from Sphingomonas sp. (HXN-200).

11. The method according to claim 1 wherein the enzyme is from an enzyme group selected from the group consisting of oxidoreductases, transferases, hydrolases, lyases, isomerases, ligases, and translocases.

12. A method of preparing a polymer-functionalized welled plate for enzyme immobilization comprising the steps of:
depositing a solution of bis[bromo]benzophenone (2BrBP) in toluene into the wells of the plate, whereby the toluene causes the well material to swell which allows the 2BrBP to intercalate into the walls of the plate;
irradiating the wells of the plate with UV-light to crosslink the 2BrBP;
depositing a mixture of monomers in the wells of the plate, wherein the mixture of monomers in a plurality of the wells systematically varies in relative proportion across a series of the wells; and
irradiating the wells of the plate with green light to initiate the polymerization of the copolymer brushes.

13. The method according to claim 12 wherein the wells are polypropylene, polystyrene or polycarbonate.

14. The method according to claim 12 wherein the copolymer brush is formed from an aromatic monomer in combination with a non-aromatic monomer, wherein the co-polymer brush is adapted to stabilize an enzyme to be linked thereto.

15. The method according to claim 14 wherein the aromatic monomer is selected from the group consisting of ethylene glycol phenyl ether methacrylate (EGPMA), phenyl methacrylate, benzyl methacrylate, diethylene glycol phenyl ether methacrylate, triethylene glycol phenyl ether methacrylate, tetraethylene glycol phenyl ether methacrylate, pentaethylene glycol phenyl ether methacrylate, poly(ethylene glycol) phenyl ether methacrylate, 1-Naphthyl methacrylate, 2-Naphthyl methacrylate, 1-Pyrenemethyl methacrylate, 9-Anthracenemethyl methacrylate, styrene, ethylene glycol phenyl ether acrylate, phenyl acrylate, benzyl acrylate, diethylene glycol phenyl ether acrylate, triethylene glycol phenyl ether acrylate, tetraethylene glycol phenyl ether acrylate, pentaethylene glycol phenyl ether acrylate, poly(ethylene glycol) phenyl ether acrylate, 1-Naphthyl acrylate, 2-Naphthyl acrylate, 1-Pyrenemethyl acrylate, 9-Anthracenemethyl acrylate, ethylene glycol phenyl ether acrylamide, phenyl acrylamide, benzyl acrylamide, diethylene glycol phenyl ether acrylamide, triethylene glycol phenyl ether acrylamide, tetraethylene glycol phenyl ether acrylamide, pentaethylene glycol phenyl ether acrylamide, poly(ethylene glycol) phenyl ether acrylamide, 1-Naphthyl acrylamide, 2-Naphthyl acrylamide, 1-Pyrenemethyl acrylamide and 9-Anthracenemethyl acrylamide, ethylene glycol phenyl ether methacrylamide, phenyl methacrylamide, benzyl methacrylamide, diethylene glycol phenyl ether methacrylamide, triethylene glycol phenyl ether methacrylamide, tetraethylene glycol phenyl ether methacrylamide, pentaethylene glycol phenyl ether methacrylamide, poly(ethylene glycol) phenyl ether methacrylamide, 1-Naphthyl methacrylamide, 2-Naphthyl methacrylamide, 1-Pyrenemethyl methacrylamide and 9-Anthracenemethyl methacrylamide.

16. The method according to claim 14 wherein the non-aromatic monomer is selected from the group consisting of sulfobetaine methacrylate (SBMA), Phosphorylcholine methacrylate (PCMA), Glycosyloxyethyl methacrylate (GEMA), poly(ethylene glycol) methacrylate (PEGMA).

17. The method according to claim 14 wherein the non-aromatic monomer is selected from the group consisting of [3-(methacryloylamino)propyl]trimethylammonium chloride, [2-(methacryloyloxy)ethyl)]trimethylammonium chloride, 3-sulfopropyl methacrylate, 3-sulfopropyl acrylate, 3-sulfopropyl acrylamide, 3-sulfopropyl methacrylamide, [2-(methacryloyloxy)ethyl]dimethyl-(3-sulfopropyl)ammonium hydroxide, methacrylic acid, acrylic acid, acrylamide, methacrylamide, poly(ethylene glycol) methacrylate, poly(ethylene glycol) acrylate, poly(ethylene glycol) acrylamide, poly(ethylene glycol) methacrylamide, 2-(diethylamino)ethyl methacrylate, 2-(diethylamino)ethyl acrylate, 2-(diethylamino)ethyl acrylamide, 2-(diethylamino)ethyl methacrylamide, n-isopropylacrylamide, 2-N-morpholinoethyl methacrylate, 2-N-morpholinoethyl acrylate, 2-N-morpholinoethyl acrylamide, and 2-N-morpholinoethyl methacrylamide.

18. The method according to claim 14 wherein the non-aromatic monomer is a zwitterionic monomer.

19. The method according to claim 18 wherein the zwitterionic monomer is selected from the group consisting of sulfobetaine acrylate, sulfobetaine acrylamide, sulfobetaine methacrylamide, phosphorylcholine acrylate, phosphorylcholine acrylamide, phosphorylcholine methacrylamide, acryloyl serine, acryloyl ornithine, acryloyl lysine, and acryloyl glutamate.

20. The method according to claim 12 further comprising the step of depositing an enzyme into the wells after copolymer brush polymerization, whereby the enzyme attaches to the copolymer brush.

21. The method according to claim 20 wherein the enzyme is selected from the groups lipase, carbonic anhydrase, cytochrome P450, benzaldehyde lyase, alkaline phosphatase, trypsin, chymotrypsin, thrombin, subtilisin, horseradish peroxidase, acetylcholinesterase, glucose isomerase, penicillin g acylase, epimerase, phytase, protein A, transaminase, nitroreductase, unspecific peroxygenase, and imine reductase.

22-32. (canceled)

33. A method to rapidly screen multi-component random copolymer compositions as supports for enzymes comprising the steps of:
functionalizing the walls of the well of a polypropylene (PP) well or surface with bis[bromo]benzophenone (2BrBP) by depositing a solution of 2BrBP in toluene on the wells or surface, wherein the toluene caused the PP to swell, allowing 2BrBP to intercalate into the well or surface;
cross-linking the 2BrBP and PP by exposing the wells to UV irradiation;
contacting the crosslinked PP-2BrBP with solutions comprising varying concentrations of a plurality of monomers;
forming copolymers by reacting the solutions comprising varying concentrations of a plurality of monomers using a green-light-activated SI-ATRP method; and
immobilizing an enzyme on the resulting copolymer brush.

34. The method to rapidly screen multi-component random copolymer compositions as supports for enzymes according to claim 33, further comprising the step of measuring the relative activity of the enzymes in each well of the plate following the immobilizing step, wherein a higher activity demonstrates an enhanced support of the enzyme by the copolymer in the well.

35-47. (canceled)
